(19) United States
(12) Patent Application Publication
(10) Pub. No.: US 2023/0082431 A1
(43) Pub. Date: Mar. 16, 2023

(54) MULTIVARIATE SPIROMETER, ASSOCIATED SYSTEMS AND METHODS

(71) Applicant: Klick Inc., Toronto (CA)

(72) Inventors: Yan Fossat, Toronto (CA); Anirudh Thommandram, Toronto (CA); Jessica Lynn Oreskovic, Richmond Hill (CA)

(21) Appl. No.: 17/902,264

(22) Filed: Sep. 2, 2022

Related U.S. Application Data
(60) Provisional application No. 63/240,089, filed on Sep. 2, 2021.

(51) Int. Cl. A61B 5/087; A61B 5/083

(52) U.S. Cl. CPC A61B 5/0878; A61B 5/0836

(57) ABSTRACT

A multivariate spirometer and associated systems and methods are described. The spirometer has a housing defining a flow path and one or more sensors for detecting CO₂ concentration values, temperature and/or flow rates of air in the flow path. The spirometer may be configured to generate time series data by sampling CO₂ concentration values, temperature and/or flow rates at a predetermined frequency during inhalation and/or exhalation. Optionally, a processor is used to generate one or more output readings based on the CO₂ concentration values, temperatures and/or flow rates.

### CROSS-REFERENCE TO RELATED APPLICATIONS

This application claims the benefit of U.S. provisional Application No. 63/240,089 filed Sep. 2, 2021 which is incorporated herein by reference in its entirety.

### FIELD

The described embodiments relate to spirometry and more specifically to multivariate spirometry.

### BACKGROUND

Spirometry is a common pulmonary function test for measuring lung function. Generally, spirometry refers to the measurement of the amount (volume) and/or speed (flow) of air that is inspired (inhaled) and/or expired (exhaled). Spirometry may be helpful for assessing pulmonary function in relation to bronchial asthma and chronic obstructive pulmonary disease and other respiratory disorders.

The peak expiratory flow (PEF) is an example of a spirometric value indicative of a person's maximum expiration flow rate. This value is typically measured with a peak flow meter, a small, hand-held device used to monitor a person's ability to breathe out air. This spirometric value however, is a peak value and does not provide for an analysis of time-varying characteristics of breathing.

There remains a need for devices, systems and methods for the measurement of variables associated with breathing.

### SUMMARY

Embodiments are provided for a spirometer apparatus, system, and method. In one aspect, the spirometer has a plurality of sensors, including a flow sensor, a temperature sensor, and a carbon dioxide sensor (CO₂). During a breathing including an inhalation and/or an exhalation, the spirometer may measure the flow rate, rate of flow rate change, temperature, rate of temperature change, CO₂ concentration, and rate of CO₂ concentration change. The measurements may be made at a configurable frequency and provide for time series data useful for analysis of a subject's breathing and/or lung function.

In a first aspect there is provided a spirometer comprising a housing comprising a first opening in fluid communication with a second opening, the first opening and second opening defining a flow path and one or more sensors selected from the group of a carbon dioxide (CO₂) sensor for detecting CO₂ concentration values of air in the flow path, a temperature sensor for detecting temperature of air in the flow path and a flow sensor for determining flow rates of air in the flow path.

In one or more embodiments, the spirometer may comprise two or more sensors selected from the CO₂ sensor, the temperature sensor and the flow sensor.

In one or more embodiments, the spirometer may comprise the CO₂ sensor, the temperature sensor and the flow sensor.

In one or more embodiments, the temperature sensor comprises a temperature sensing member proximate to the first opening in the flow path relative to the CO₂ sensor and/or the flow sensor, optionally relative to a CO₂ sensing member and/or a flow sensing member.

In one or more embodiments, the flow path may be linear. Alternatively, the flow path may be non-linear.

In one or more embodiments, the one or more sensors may detect a plurality of CO₂ concentration values, a plurality of temperatures and/or a plurality of flow rates during inhalation of air by a subject through the flow path.

In one or more embodiments, the one or more sensors may detect a plurality of CO₂ concentration values, a plurality of temperatures and/or a plurality of flow rates during exhalation of air by a subject through the flow path.

In one or more embodiments, the one or more sensors may detect the plurality of CO₂ concentration values, the plurality of temperatures, and/or the plurality of flow rates as the subject continuously breathes through the flow path.

In one or more embodiments, the first opening may be configured to detachably receive a mouthpiece. The mouthpiece may be shaped to form a substantially airtight seal with the lips of a subject using the spirometer. For example, the mouthpiece may have a circular or oval cross section. In one or more embodiments, the mouthpiece may be made of a plastic material that is biodegradable at a temperature above 50 degrees Celsius.

In one or more embodiments, the spirometer may further comprise a laminarization means disposed between the first opening and the second opening in order to produce a laminar airflow through the flow path.

In one or more embodiments, the temperature sensor may be proximate to the first opening relative to the CO₂ sensor or the flow sensor.

In one or more embodiments, the one or more sensors may detect CO₂ concentration values, temperatures and/or flow rates at a predetermined frequency of at least 3, 4, 5, 6, 7, 8, 9, 10, 20, 25, 30, 40 or 50 times per second.

In one or more embodiments, the temperature sensor may have a resolution of at least 0.5, 0.25, 0.1, 0.08, 0.06, 0.05 or 0.04 degrees Celsius.

In one or more embodiments, the flow sensor may be a bidirectional flow sensor.

In one or more embodiments, the CO₂ sensor may comprise a metal oxide gas sensor for detecting volatile organic compounds (VOCs), optionally an ams® Digital Gas Sensor. In one or more embodiments the temperature sensor may comprise a band gap temperature sensor, optionally a Microchip® Digital Temperature Sensor. In one or more embodiments, the flow sensor may comprise a thermal mass flow sensor element, optionally a Sensiron® Digital Flow Meter Sensor.

In one or more embodiments, the spirometer may further comprise a real-time clock. In one or more embodiments, the one or more sensors may generate a plurality of sensor readings timestamped with an output of the real-time clock.

In one or more embodiments, the spirometer may further comprise a processor for generating one or more output readings based on the one or more CO₂ concentration values, temperatures and/or flow rates detected by the one or more sensors.

In one or more embodiments, the spirometer may further comprise memory for storing the one or more CO₂ concentration values, temperatures and/or flow rates detected by the one or more sensors, optionally for storing the one or more output readings.

In one or more embodiments, the spirometer may further comprise a display for presenting the output readings to a user.

In a second aspect, there is provided a spirometer system. In one embodiment, the system comprises a spirometer comprising a first opening in fluid communication with a second opening, the first opening and second opening defining a flow path, one or more sensors, a processor, and a memory. In one embodiment, the one or more sensors are selected from a CO₂ sensor for sensing a plurality of CO₂ concentration values of air in the flow path, a temperature sensor for sensing a plurality of temperature values of air in the flow path, and/or a flow rate sensor for sensing a plurality of flow rate values of air in the flow path. In one embodiment, the processor is in communication with the memory and the processor configured to receive a plurality of measured sensor values from the CO₂ sensor, the temperature sensor and/or the flow rate sensor, determine one or more output values based on the plurality of measured sensor values and store, at the memory, the one or more output values.

In one or more embodiments, the spirometer may comprise the CO₂ sensor, the temperature sensor and the flow rate sensor. In one embodiment, the plurality of measured sensor values may comprise or consist of a plurality of CO₂ concentration values, a plurality of temperature values and a plurality of flow rate values.

In one or more embodiments, the processor receives the plurality of measured sensor values at a predetermined frequency over a data collection period. The plurality of measured sensor values and the plurality of output sensor values may be time series data.

In one or more embodiments, the predetermined frequency may be at least 2 Hz, 3 Hz, 4 Hz, 5 Hz or 10 Hz and the data collection period is at least 5, 10, 15, 20 or 30 seconds, optionally at least 2, 3, 4, 5 subject breaths.

In one or more embodiments, the receiving, at the processor, a first one or more of the group of the plurality of CO₂ concentration values, the plurality of temperature values, and the plurality of flow rate values may occur at a first predetermined frequency over a data collection period; the receiving, at the processor, a second one or more of the group of the plurality of CO₂ concentration values, the plurality of temperature values, and the plurality of flow rate values may occur at a second predetermined frequency over a data collection period, and the plurality of measured sensor values and the plurality of output sensor values may be time series data.

In one or more embodiments, the spirometer device may further comprise a button for sending a user input signal to the processor; and wherein the processor may be configured to perform the steps of receiving, determining, and storing in response to the user input signal.

In one or more embodiments, the processor may be configured to: determine one or more of the group of: a plurality of first determined temperature values based on the plurality of output sensor values, wherein the plurality of first determined temperature values are a function of instantaneous air flow; one or more inflection points of the plurality of first determined temperature values; a plurality of second determined temperature values based on the plurality of first determined temperature values, wherein the second determined temperature values are a function of percentage of air exhaled; one or more inflection points of the plurality of second determined temperature values; a plurality of first determined CO₂ concentration values, based on the plurality of output sensor values, wherein the plurality of first determined CO₂ concentration values are a function of instantaneous air flow; one or more inflection points of the plurality of first determined CO₂ concentration values; a plurality of second determined CO₂ concentration values based on the plurality of first determined temperature values, wherein the second determined CO2 concentration values are a function of percentage of air exhaled; and one or more inflection points of the plurality of second determined CO₂ concentration values.

In one or more embodiments, the plurality of first determined temperature values may be based on a derivative of the plurality of output sensor values; the plurality of second determined temperature values may be based on a derivative of the plurality of first determined temperature values; the plurality of first determined CO₂ concentration values may be based on a derivative of the plurality of output sensor values; and the plurality of second determined CO₂ concentration values may be based on a derivative of the plurality of first determined temperature values.

In one or more embodiments, the first derivative temperature values, second derivative temperature values, first derivative CO₂ concentration values, and second derivative CO₂ concentration values may correspond to the time series of the plurality of output sensor values.

In one or more embodiments, the processor may be further configured to:

collect a plurality of baseline sensor values; determining the plurality of calibration values from the plurality of baseline sensor values; and wherein the processor may determine the plurality of output values from the plurality of calibration values and the plurality of measured sensor values.

In one or more embodiments, the spirometer system may further comprise: a display device in communication with the processor; wherein the processor may be further configured to: output to the display device, the plurality of output values, the first derivative temperature values, second derivative temperature values, first derivative CO2 concentration values, and second derivative CO₂ concentration values.

In a third aspect, there is provided a method of operating a spirometer, the method comprising: receiving, at a processor, a plurality of measured sensor values; determining, at the processor, a plurality of output values from the plurality of measured sensor values; and storing, at a memory in communication with the processor, the plurality of output values.

In one or more embodiments, the plurality of measured sensor values may comprise: a plurality of CO₂ concentration values of the breath sensed by a CO₂ sensor; a plurality of temperature values sensed by a temperature sensor; and a plurality of flow rate values sensed by a flow rate sensor for sensing of the breath.

In one or more embodiments, the receiving, at the processor, the plurality of measured sensor values may occur at a predetermined frequency over a data collection period; and the plurality of measured sensor values and the plurality of output sensor values may be time series data.

In one or more embodiments, the predetermined frequency may be at least 2, Hz, 3 Hz, 4 Hz, 5 Hz or 10 Hz and the data collection period may be at least 5, 10, 15, 20 or 30 seconds, optionally at least 2, 3, 4, 5 subject breaths.

In one or more embodiments, the receiving, at the processor, a first one or more of the group of the plurality of CO₂ concentration values, the plurality of temperature values, and the plurality of flow rate values may occur at a first predetermined frequency over a data collection period; the receiving, at the processor, a second one or more of the group of the plurality of CO₂ concentration values, the plurality of temperature values, and the plurality of flow rate values may occur at a second predetermined frequency over a data collection period, the plurality of measured sensor values and the plurality of output sensor values may be time series data.

In one or more embodiments, the method may further comprise: receiving, at the processor, a user input signal from a button; and wherein, in response to receiving the user input, the processor performs the steps of receiving, determining, and storing.

In one or more embodiments, the method may further comprise: determining, at the processor, one or more of the group of: a plurality of first determined temperature values based on the plurality of output sensor values, wherein the plurality of first determined temperature values are a function of instantaneous air flow; one or more inflection points of the plurality of first determined temperature values; a plurality of second determined temperature values based on the plurality of first determined temperature values, wherein the second determined temperature values are a function of percentage of air exhaled; one or more inflection points of the plurality of second determined temperature values; a plurality of first determined CO₂ concentration values, based on the plurality of output sensor values, wherein the plurality of first determined CO₂ concentration values are a function of instantaneous air flow; one or more inflection points of the plurality of first determined CO₂ concentration values; a plurality of second determined CO₂ concentration values based on the plurality of first determined temperature values, wherein the second determined CO₂ concentration values are a function of percentage of air exhaled; and one or more inflection points of the plurality of second determined CO₂ concentration values.

In one or more embodiments, the plurality of first determined temperature values may be based on a derivative of the plurality of output sensor values; the plurality of second determined temperature values may be based on a derivative of the plurality of first determined temperature values; the plurality of first determined CO₂ concentration values may be based on a derivative of the plurality of output sensor values; the plurality of second determined CO₂ concentration values may be based on a derivative of the plurality of first determined temperature values.

In one or more embodiments, the first derivative temperature values, second derivative temperature values, first derivative CO₂ concentration values, and second derivative CO₂ concentration values may correspond to the time series of the plurality of output sensor values.

In one or more embodiments, the method may further comprise: collecting, at the processor, a plurality of baseline sensor values; determining, at the processor, the plurality of calibration values from the plurality of baseline sensor values; and wherein the determining, at the processor, the plurality of output values is further based on the plurality of calibration values and the plurality of measured sensor values.

In one or more embodiments, the method may further comprise: outputting, at a display device, the plurality of output values, the first derivative temperature values, second derivative temperature values, first derivative CO₂ concentration values, and second derivative CO₂ concentration values.

### BRIEF DESCRIPTION OF THE DRAWINGS

A preferred embodiment of the present invention will now be described in detail with reference to the drawings, in which:

FIG. 1 shows a rear perspective view diagram of a spirometer.

FIG. 2 shows a front perspective view diagram of the spirometer.

FIG. 3 shows a right side view diagram of the spirometer.

FIG. 4 shows a left side view diagram of the spirometer.

FIG. 5 shows a rear side view diagram of the spirometer.

FIG. 6 shows a front side view diagram of the spirometer.

FIG. 7 shows a top plan view diagram of the spirometer.

FIG. 8 shows a bottom side view diagram of the spirometer.

FIG. 9 shows an exploded rear perspective view diagram of the spirometer.

FIG. 10 shows an exploded front perspective view diagram of the spirometer.

FIG. 11 shows an exploded right side view diagram of the spirometer.

FIG. 12 shows an exploded left side view diagram of the spirometer.

FIG. 13 shows an exploded top plan view diagram of the spirometer.

FIG. 14 shows an exploded bottom side view diagram of the spirometer.

FIG. 15 shows a front perspective diagram of a mouthpiece of the spirometer.

FIG. 16 shows a rear perspective diagram of the mouthpiece of the spirometer.

FIG. 17 shows a left side view diagram of the mouthpiece of the spirometer.

FIG. 18 shows a front view diagram of the mouthpiece of the spirometer.

FIG. 19 shows a right side view diagram of the mouthpiece of the spirometer.

FIG. 20 shows a rear side view diagram of the mouthpiece of the spirometer.

FIG. 21 shows a top plan view diagram of the mouthpiece of the spirometer.

FIG. 22 shows a bottom side view diagram of the mouthpiece of the spirometer.

FIG. 23 shows a user interface diagram of the spirometer.

FIG. 24 shows a system diagram of the spirometer.

FIG. 25 shows a block diagram of the spirometer.

FIG. 26 shows a method diagram of the spirometer.

FIG. 27 shows an example output of the spirometer.

FIG. 28 shows an example respiration cycle with flow rate and CO₂ content in Example 1.

FIG. 29a shows results from Example 1 of Second Breath Max Temperature for Uncontrolled vs. Controlled Asthma groups.

FIG. 29b shows results from Example 1 of Time to CO₂ increase for Uncontrolled vs. Controlled Asthma groups.

FIG. 29c shows results from Example 1 of Volume Exhale After CO₂ Increase for Uncontrolled vs. Controlled Asthma groups.

FIG. 29d shows results from Example 1 of Volume Exhale After Temperature Increase for Uncontrolled vs. Controlled Asthma groups.

FIG. 29e shows results from Example 1 of Volume Exhale After CO₂ Increase for Uncontrolled vs. Controlled Asthma groups.

FIG. 29f shows results from Example 1 of Volume After CO₂ Increase (Volume After Temp Increase) for Uncontrolled vs. Controlled Asthma groups.

FIG. 29g shows results from Example 1 of Inhale/Exhale Switch for Uncontrolled vs. Controlled Asthma groups.

FIG. 29h shows results from Example 1 of Exhale Proportion for Uncontrolled vs. Controlled Asthma groups.

FIG. 29i shows results from Example 1 of V1/Vtotal for Uncontrolled vs. Controlled Asthma groups.

FIG. 29j shows results from Example 1 of V2/Vtotal for Uncontrolled vs. Controlled Asthma groups.

FIG. 30a shows results from Example 1 of Volume Exhaled after Temperature Increase for Asymptomatic vs. Symptomatic groups.

FIG. 30b shows results from Example 1 of Volume Exhaled after CO₂ Increase for Asymptomatic vs. Symptomatic groups.

FIG. 30c shows results from Example 1 Maximum Temperature Second Breath for Asymptomatic vs. Symptomatic groups.

FIG. 30d shows results from Example 1 of Time to Temperature Increase for Asymptomatic vs. Symptomatic groups.

FIG. 30e shows results from Example 1 of Breathing Rate for Asymptomatic vs. Symptomatic groups.

FIG. 30f shows results from Example 1 of Inhale-Exhale Switch for Asymptomatic vs. Symptomatic groups.

FIG. 30g shows results from Example 1 of V1/Vtotal for Asymptomatic vs. Symptomatic groups.

FIG. 30h shows results from Example 1 of V2/Vtotal for Asymptomatic vs. Symptomatic groups.

FIG. 30i shows results from Example 1 of Volume Exhaled Product for Asymptomatic vs. Symptomatic groups.

FIG. 30j shows results from Example 1 of Time to CO₂ Increase for Asymptomatic vs. Symptomatic groups.

FIG. 30k shows results from Example 1 of Exhale Proportion for Asymptomatic vs. Symptomatic groups.

FIG. 30l shows results from Example 1 of T1/T2 for Asymptomatic vs. Symptomatic groups.

FIG. 31a shows results from Example 1 of Breathing Rate for Healthy vs. Asthmatic groups.

FIG. 31b shows results from Example 1 of V1/V2 for Healthy vs. Asthmatic groups.

FIG. 31c shows results from Example 1 V1/Vtotal for Healthy vs. Asthmatic groups.

FIG. 31d shows results from Example 1 of V2/Vtotal for Healthy vs. Asthmatic groups.

FIG. 32a shows results from Example 1 of Breathing Rate for Healthy Group vs. Symptomatic Asthma Group.

FIG. 32b shows results from Example 1 of V1/V2 for Healthy Group vs. Symptomatic Asthma Group.

FIG. 32c shows results from Example 1 V1/Vtotal for Healthy Group vs. Symptomatic Asthma Group.

FIG. 32d shows results from Example 1 of V2/Vtotal for Healthy Group vs. Symptomatic Asthma Group.

FIG. 32e shows results from Example 1 of Time to CO2 Increase for Healthy Group vs. Symptomatic Asthma Group.

FIG. 32f shows results from Example 1 of Area Flow After CO2 Rise (V2) for Healthy Group vs. Symptomatic Asthma Group.

FIG. 32g shows results from Example 1 Area Flow After Temperature Rise (T2) for Healthy Group vs. Symptomatic Asthma Group.

FIG. 32h shows results from Example 1 of Inhale/Exhale Switch for Healthy Group vs. Symptomatic Asthma Group.

### DESCRIPTION OF EXEMPLARY EMBODIMENTS

It will be appreciated that numerous specific details are set forth in order to provide a thorough understanding of the example embodiments described herein. However, it will be understood by those of ordinary skill in the art that the embodiments described herein may be practiced without these specific details. In other instances, well-known methods, procedures and components have not been described in detail so as not to obscure the embodiments described herein. Furthermore, this description and the drawings are not to be considered as limiting the scope of the embodiments described herein in any way, but rather as merely describing the implementation of the various embodiments described herein.

It should be noted that terms of degree such as “substantially”, “about” and “approximately” when used herein mean a reasonable amount of deviation of the modified term such that the end result is not significantly changed. These terms of degree should be construed as including a deviation of the modified term if this deviation would not negate the meaning of the term it modifies.

In addition, as used herein, the wording “and/or” is intended to represent an inclusive-or. That is, “X and/or Y” is intended to mean X or Y or both, for example. As a further example, “X, Y, and/or Z” is intended to mean X or Y or Z or any combination thereof.

The embodiments of the systems and methods described herein may be implemented in hardware or software, or a combination of both. These embodiments may be implemented in computer programs executing on programmable computers, each computer including at least one processor, a data storage system (including volatile memory or non-volatile memory or other data storage elements or a combination thereof), and at least one communication interface. For example and without limitation, the programmable computers (referred to below as computing devices) may be a server, network appliance, embedded device, computer expansion module, a personal computer, laptop, personal data assistant, cellular telephone, smart-phone device, tablet computer, a wireless device or any other computing device capable of being configured to carry out the methods described herein.

In some embodiments, the communication interface may be a network communication interface. In embodiments in which elements are combined, the communication interface may be a software communication interface, such as those for inter-process communication (IPC). In still other embodiments, there may be a combination of communication interfaces implemented as hardware, software, and combination thereof.

Program code may be applied to input data to perform the functions described herein and to generate output information. The output information is applied to one or more output devices, in known fashion.

Each program may be implemented in a high level procedural or object oriented programming and/or scripting language, or both, to communicate with a computer system. However, the programs may be implemented in assembly or machine language, if desired. In any case, the language may be a compiled or interpreted language. Each such computer program may be stored on a storage media or a device (e.g. ROM, magnetic disk, optical disc) readable by a general or special purpose programmable computer, for configuring and operating the computer when the storage media or device is read by the computer to perform the procedures described herein. Embodiments of the system may also be considered to be implemented as a non-transitory computer-readable storage medium, configured with a computer program, where the storage medium so configured causes a computer to operate in a specific and predefined manner to perform the functions described herein.

Furthermore, the system, processes and methods of the described embodiments are capable of being distributed in a computer program product comprising a computer readable medium that bears computer usable instructions for one or more processors. The medium may be provided in various forms, including one or more diskettes, compact disks, tapes, chips, wireline transmissions, satellite transmissions, internet transmission or downloads, magnetic and electronic storage media, digital and analog signals, and the like. The computer useable instructions may also be in various forms, including compiled and non-compiled code.

Various embodiments have been described herein by way of example only. Various modification and variations may be made to these example embodiments without departing from the spirit and scope of the invention, which is limited only by the appended claims. Also, in the various user interfaces illustrated in the figures, it will be understood that the illustrated user interface text and controls are provided as examples only and are not meant to be limiting. Other suitable user interface elements may be possible.

Many spirometers are useful for determining peak flow rates for subject's breathing. The breathing indicators of a subject, however, can more broadly include the changes in air flow, temperature, and CO₂ concentration in a breath over time.

Present embodiments related to the measurement of air in a subject's breath that includes indicators of gas that is found deeper in the lungs. Instead of looking simply to single values, time-series data is determined to identify the different temperature, air flow, and CO₂ concentrations of gas in a patient's breath through their entire inhalation/exhalation cycle, and over the course of several breaths. Such measurements may include the time series sensor data, along with determined values from the time series data. The measured values therefore include the measurement of temperature, CO₂ concentration, and flow rate of air that is found deeper in the lungs, which may measure physiological differences of the subject not available using conventional spirometers. These physiological differences may include sickness, recovery of lung transplant patients, etc.

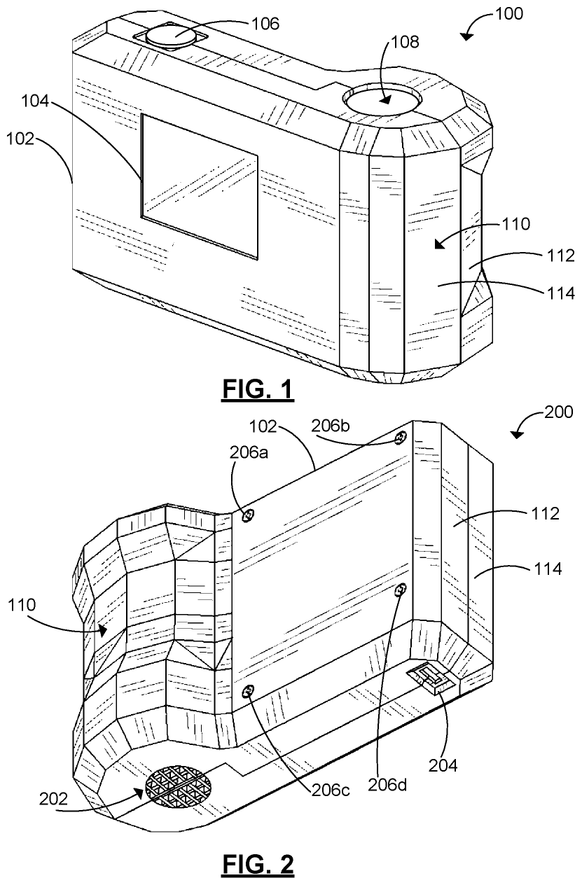

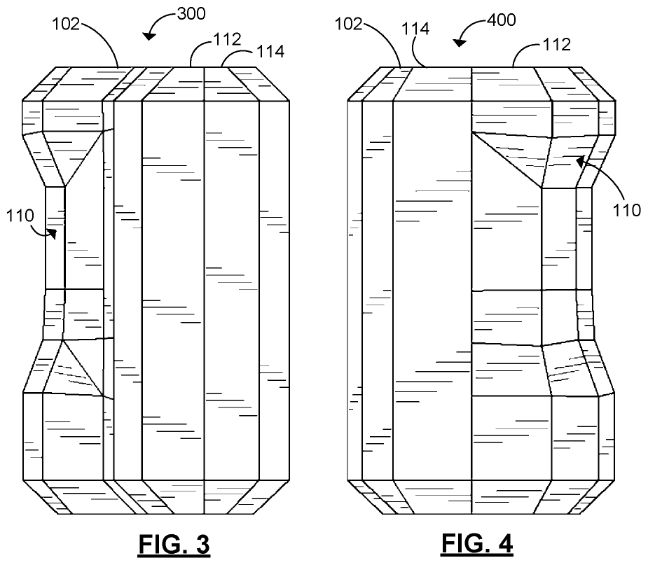

Reference is first made to FIGS. 1, 2, 3, 4, 5, 6, 7, and 8 together, showing various diagrams of a spirometer according to at least one embodiment.

The spirometer has an enclosure 102 having a user input device 106, a display 104, a data port 204, a first opening 108, and a second opening 202. The enclosure may be generally rectangular including a grip portion 110.

The spirometer enclosure 102 may be formed as a single piece, or in a preferred embodiment may be formed from a front piece 112 and a back piece 114. The front piece 112 and the back piece 114 may be attached using a plurality of fasteners 206. The fasteners may be screws, locking tabs, bolts, or any other fastener as known. The front piece 112 and the back piece 114 may alternatively be glued together along their seam.

Inside the spirometer enclosure 102, there may be one or more sensors (not shown), a processor (not shown), a real time clock (not shown), and a power means (not shown).

The display 104 may include an input device such as a touchscreen. The display 104 may be, for example, a thin-film transistor (TFT) touchscreen display. A TFT touch screen is a combination device that includes a TFT liquid-crystal display (LCD) display and a touch technology overlay on the screen. The TFT device can both display content and act as an input interface device for the user: The spirometer display, when configured using a TFT may include user input via the touchscreen and output of collected data along with data and statistics determined from the collected data.

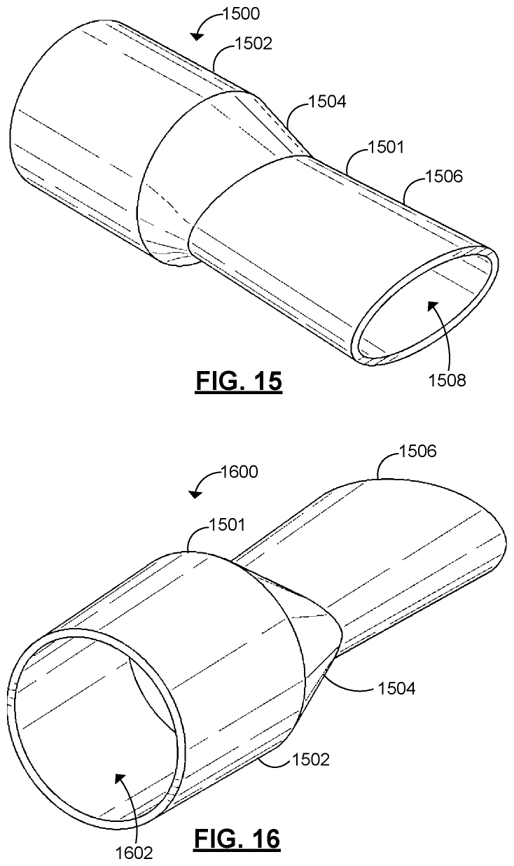

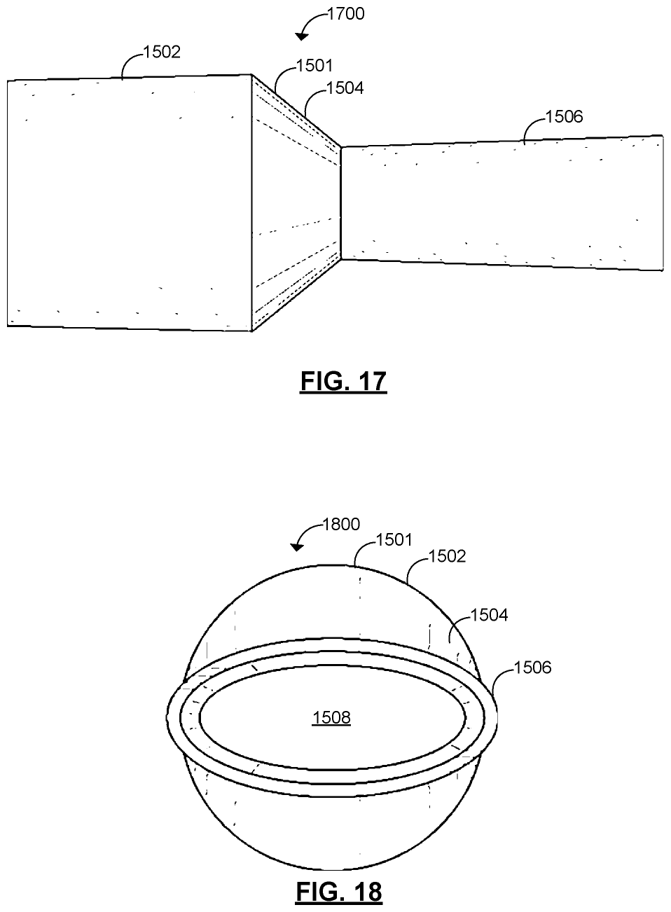

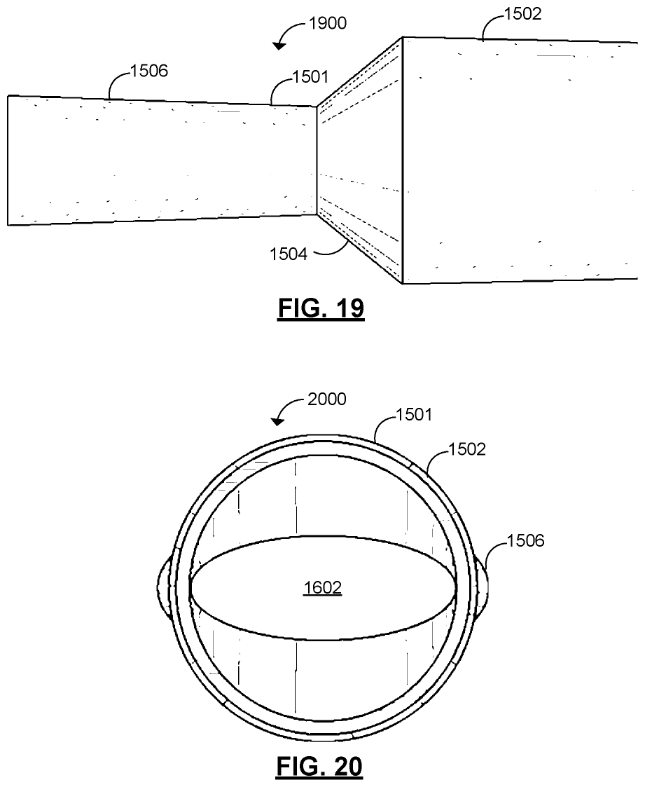

The spirometer enclosure 102 has a first opening 108 positioned proximate to a subject's mouth. The subject may position the first opening 108 on their lips and breathe directly via the first opening 108 into the spirometer. In a preferred embodiment, the first opening 108 may detachably receive a mouthpiece similar to the one shown in FIGS. 15, 16, 17, 18, 19, 20, 21, and 22, and the subject may place the mouthpiece against their lips. The first opening 108 may be sized to allow generally laminar flow of the subject's breath through the spirometer.

A second opening 202 may be positioned generally opposite the first opening 108. The second opening 202 may be sized to allow generally laminar flow of the subject's breath through the spirometer.

A button or other responsive element for generating an input signal 106 may be positioned on enclosure 102. The button 106 may provide a user input signal in order to operate one or more functions of the spirometer.

A grip portion 110 may be provided on the enclosure to provide a surface for the subject to hold in their hand while operating the spirometer.

The enclosure may have a data port 204 for downloading collected sensor data and statistics determined from the collected sensor data. The data port 204 may be a removable storage device slot, such as a Secure Digital (SD) format, Secure Digital High Capacity (SDHC) format, Secure Digital eXtended Capacity (SDXC) format, or Secure Digital Ultra Capacity (SDUC) format. The data port 204 may be a serial bus connection, such as a Universal Serial Bus (USB-A), USB-B, or USB-C style connector that may allow a computer or other device to be connected to the spirometer in order to download data. The USB connection may also provide power to spirometer and/or charge the battery inside (not shown).

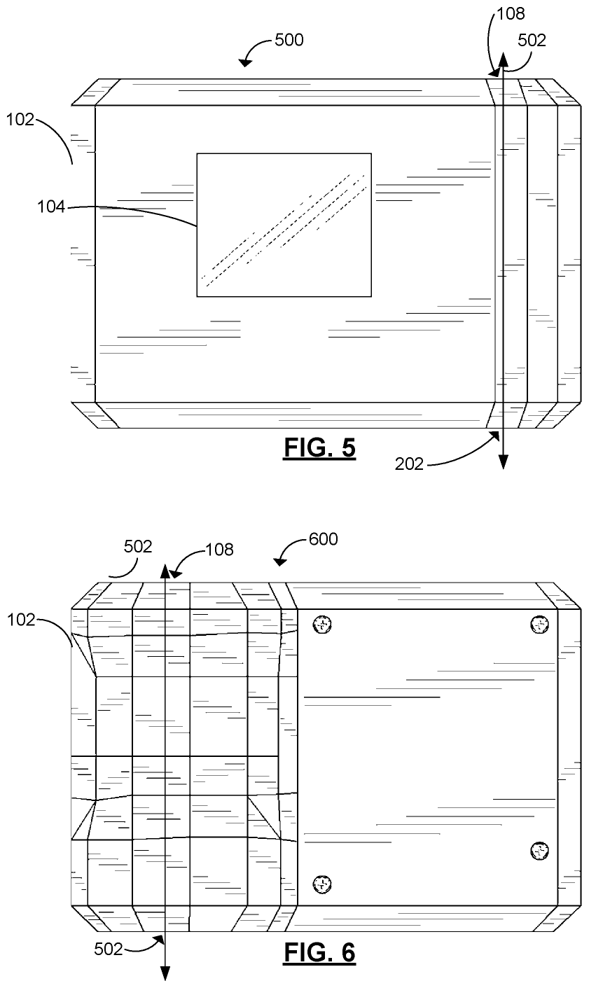

Referring to FIGS. 5 and 6 together, the flow path of a subject's breaths through the spirometer is shown. The subject's breath may pass through a flow path 502 of the spirometer. Air may flow through the first opening 108, along flow path 502, and through second opening 202. The one or more sensors (not shown) may be disposed generally along the flow path 502 inside the enclosure 102.

The subject's breath may include an inhalation and exhalation phase. During the inhalation phase, air may flow from the second opening 202, through flow path 502, then through the first opening 108, optionally through the mouthpiece (not shown), and into the subjects mouth. During the exhalation phase, air may flow from the subject, optionally through the mouthpiece (not shown), through the first opening 108, along the flow path 502, and through the second opening 202. One or more sensors (not shown) may be disposed inside the enclosure 102 along the flow path 502.

Referring next to FIG. 7, there is shown a top plan view diagram 700 of a top of the spirometer. A user input button 206, and a first opening 108 may be disposed on the top of enclosure 102.

The first opening 108 may include a generally circular aperture 702 in the enclosure 102. Attached within the generally circular aperture 702 may be a generally circular shim 704 which may provide a removable attachment point for the mouthpiece (not shown). The circular shim 702 may provide a substantially airtight interference fit with the mouthpiece. A grating 706 may be disposed across the opening 108. The grating 706 may be a means for improving the laminar airflow through the flow path. The grating 706 may be a grid oriented as shown, or may be shaped in a different pattern.

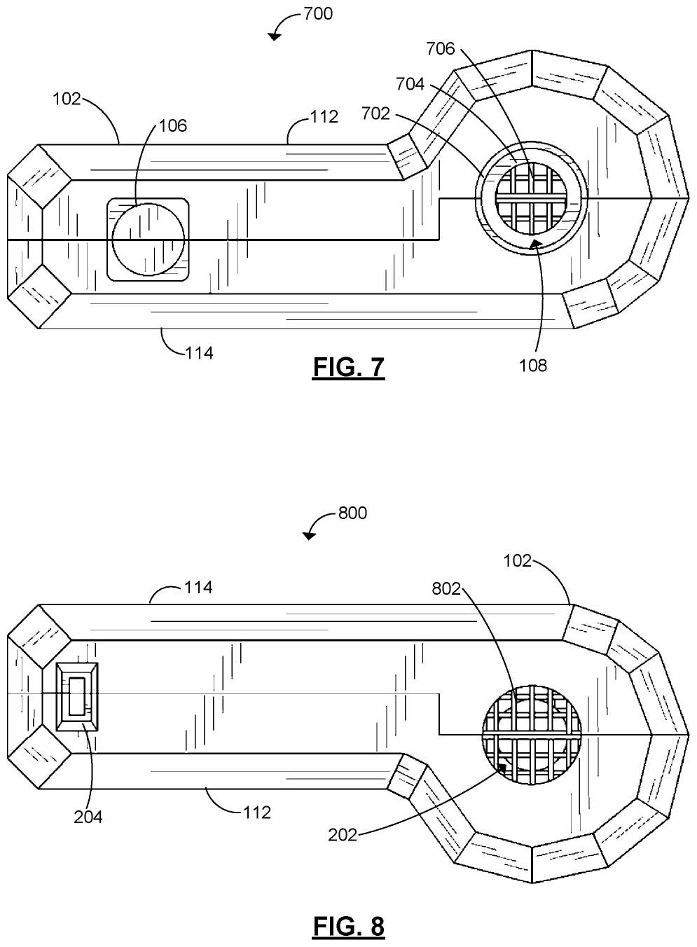

Referring next to FIG. 8, there is shown a bottom side view diagram 800 of the spirometer. A second opening 202 and a data port 204 may be disposed on the bottom of the spirometer enclosure 102.

The second opening 202 may include a grating 802 disposed across the opening 202. The grating 802 may be a means for improving the laminar airflow through the flow path. The grating 802 may be a grid oriented as shown, or may be shaped in a different pattern.

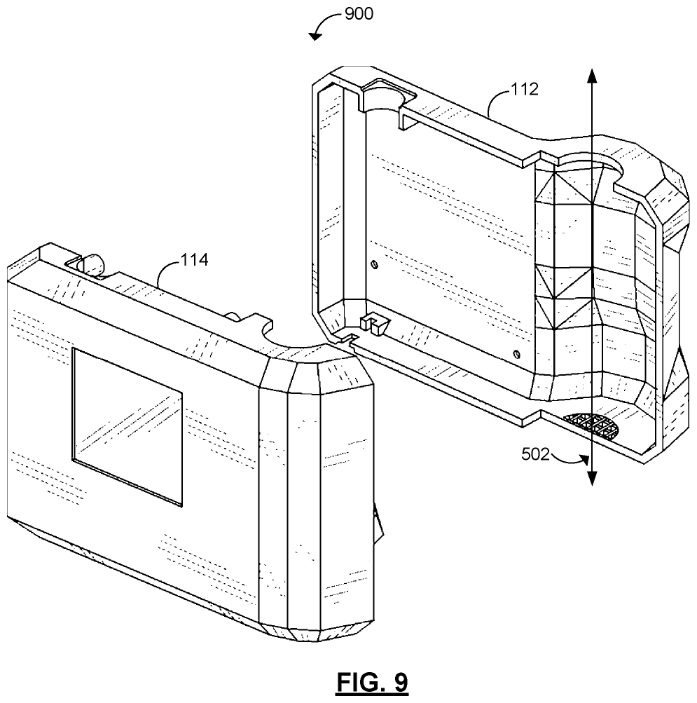

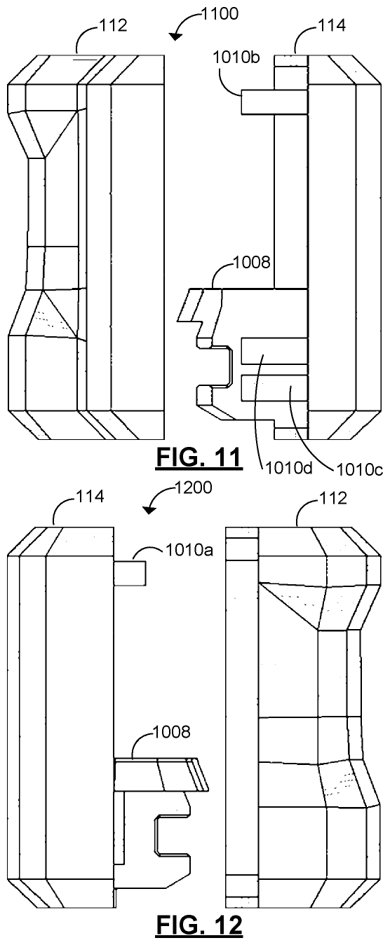

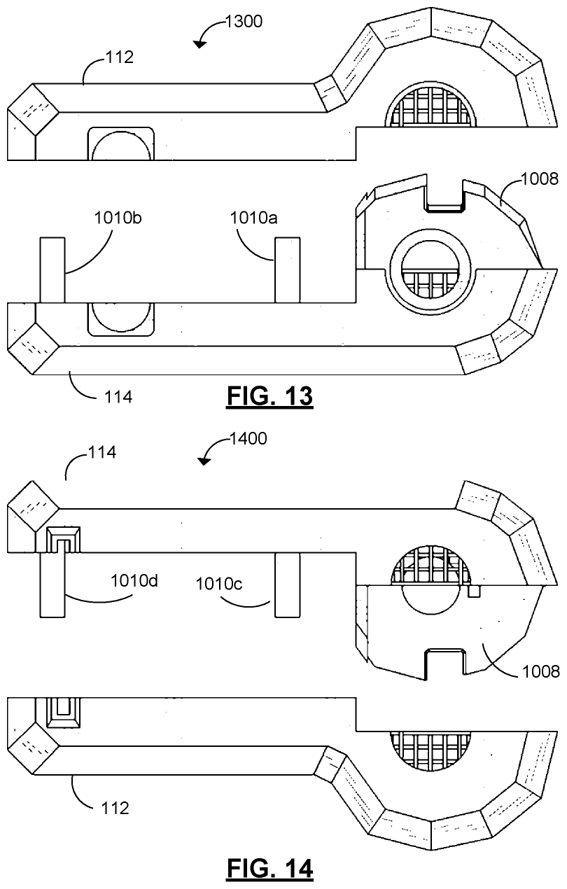

Reference is next made to FIGS. 9, 10, 11, 12, 13, and 14 together, a preferred embodiment is shown of a front piece 112 and a rear piece 114 in a separated arrangement.

Referring to FIG. 9, an exploded rear perspective view diagram 900 of the spirometer showing flow path 502 through the spirometer. The flow path 502 may be generally linear as shown, or alternative may be non-linear.

Referring next to FIG. 10, an exploded right side view diagram 1000 of the spirometer. The rear piece 114 and the front piece 112 may engage each other via protrusions 1010.

A plenum 1004 may be positioned between the air flow sensor 1006 and the second opening 202. Alternatively, plenum 1004 may be positioned between first opening 108 and air flow sensor 1006 (not shown) such that plenum 1004 is proximate to the first opening 108. The plenum may be generally defined by the back piece 114, and the baffle 1008, and may enclose one or more sensors such as the temperature sensor 1016 and/or the CO₂ sensor 1012.

The rear piece 114 may include one or more sensors disposed generally along air flow path 502, such as an air flow sensor, temperature sensor, and/or CO₂ sensor.

An air flow sensor 1006 may be positioned in air flow path 502 between first opening 108 and second opening 202. Referring to FIG. 10, in one embodiment air flow sensor 1006 is positioned proximate to first opening 108. Alternatively, air flow sensor may be positioned proximate to second opening 202 relative to other sensors in the flow path. The air flow sensor may be, for example, a SFM330-250-D from Sensirion®. In one or more embodiments, the flow sensor 1006 may have a flow capacity of +/−250 standard liters per minute (slm) or better for human breathing flow measurement. The flow sensor 1006 may be bidirectional, and may function for both a subject's inspiration and expiration through the spirometer. The flow sensor 1006 may be any type of flow sensing technology, including differential pressure sensing or thermal mass flow sensing. Preferably however, thermal mass flow sensing may be used in order to provide accurate sensing for the relatively low volume movement in regular breathing, instead of forced expiratory breathing tests used in other spirometry devices.

A temperature sensor 1012 may be positioned with temperature sensing member 1014 generally in a plenum 1004 of air flow path 502. Referring to FIG. 10, in one embodiment, temperature sensor 1012 and/or temperature sensing member 1014 is positioned proximate to second opening 202 relative to air flow sensor 1006. Alternatively, temperature sensor 1012 and/or temperature sensing member 1014 is positioned proximate to first opening 108 (not shown). In one embodiment the temperature sensor comprises a temperature sensing member proximate to the first opening 108 and/or a mouthpiece such that in operation airflow during exhalation along flow path 502 first contacts the temperature sensing member before contacting the flow sensor and/or CO₂ sensor. Without being limited by theory it is believed that the thermal mass of the device and contact with the flow sensor, CO₂ sensor and/or ambient air in the device may alter the temperature of exhaled air preventing the temperature sensor from generating accurate or consistent results. Various trials with different configurations demonstrated that placing the temperature sensing member proximate to the first opening relative to the other sensors improved sensitivity relative to other configurations (data not shown).

Temperature sensor 1012 may be a band gap temperature sensor such as an MCP9808 temperature sensor from Microchip®. The temperature sensor 1012 may have a configurable measurement resolution. Since changes in breath temperature are not very large (generally in the range of 0.5-3 degrees Celsius) the temperature sensor may have an accuracy of ±0.0625 degrees (minimum), ±0.25° C. (typical) or ±0.5° C. (maximum) which may be configurable.

A CO₂ sensor 1016 may be positioned with CO₂ sensing member 1018 generally in the plenum 1004 of air flow path 502. In one embodiment, CO₂ sensor 1016 and/or CO₂ sensing member 1018 are positioned proximate to the second opening 202 relative to air flow sensor 1006. Alternatively, CO₂ sensor 1016 and/or CO₂ sensing member 1014 is positioned proximate to first opening 108 (not shown) relative to air flow sensor 1006.

The CO₂ sensor 1016 may be a metal oxide gas sensor such as a CCS811 from amr®. The CO₂ sensor 1016 may sense CO₂ concentration directly, or indirectly. For example, the CO₂ concentration may be measured indirectly by the CO₂ sensor by detecting volatile organic compounds (VOCs). The CO₂ sensor may itself perform steps to process the raw measurements in order to output a measurement called equivalent CO₂ concentration. The CO₂ sensor may also measure or determine other VOC concentrations in addition to CO₂ concentration.

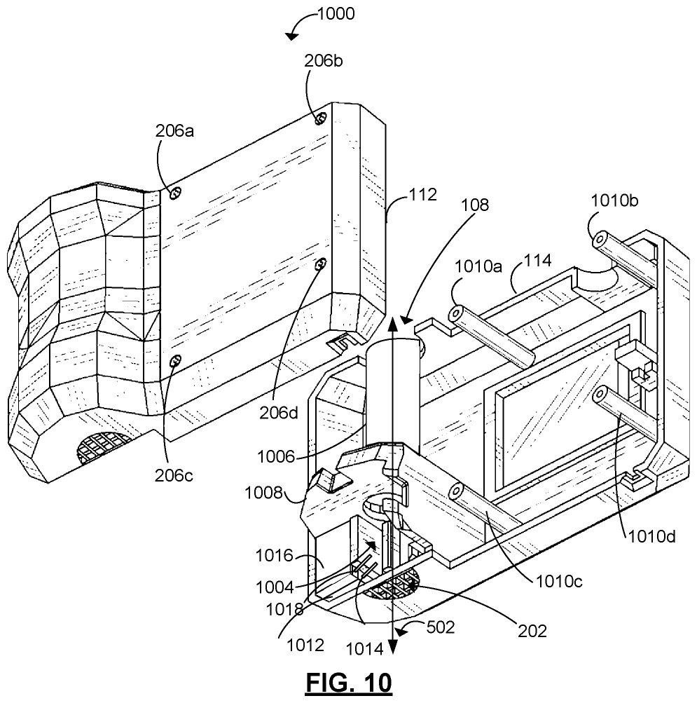

Referring next to FIGS. 11-14, the engagement of the front piece 112 and the rear piece 114 is shown, including the engagement of the baffle 1008. The front piece 112 and the rear piece 114 may engage in substantially airtight engagement. The baffle 1008 may also engage between the front piece 112 and the rear piece 114 in substantially airtight engagement. The baffle 1008, front piece 112 and rear piece 114 may be glued together, or fastened together using clips, screws, bolts, or another means of attachment. The protrusions 1010 may engage with fasteners 206 attached through the front piece 112.

Reference is next made to FIGS. 15, 16, 17, 18, 19, 20, 21, and 22 together, showing a mouthpiece for a spirometer according to at least one embodiment.

The mouthpiece 1501 may be generally cylindrical, having an oval portion 1506, a transition portion 1504, and a circular portion 1502. The oval portion 1506 is engaged with the user's lips and mouth during use, and has a first aperture 1508 for communicating the user's breath through the mouthpiece and a second aperture 1602. The transition portion 1504 may connect the oval portion 1506 and the circular portion 1502 and transition the generally circular cross section 1502 with the generally oval cross section 1506.

The oval portion 1506 may have a first diameter generally the same diameter as the circular portion 1502, and a second diameter generally smaller than the diameter of the circular portion 1502. The oval portion 1506 may have a variety of sizes of the first and second diameter in order to accommodate the different sizes and shapes of subject's mouths. While shown as generally oval, the oval portion 1506 may have another shape, such as a rectangle.

The circular portion 1502 is for removable engagement with the first opening of the spirometer. The circular portion 1502 may form an interference fit with the first opening of the spirometer. Each mouthpiece may be disposable, and used once per subject in order to provide a clean and hygienic surface for the user. While shown as generally oval, the circular portion 1502 may have another shape, such as a rectangle or an irregular shape suitable for forming a substantially airtight engagement with a user's lips.

The mouthpiece 1501 may be made from any material, but preferably from a biodegradable plastic. The biodegradable plastic may a high temperature biodegradable plastic, for example it may break down at temperatures of over 50 degrees Celsius.

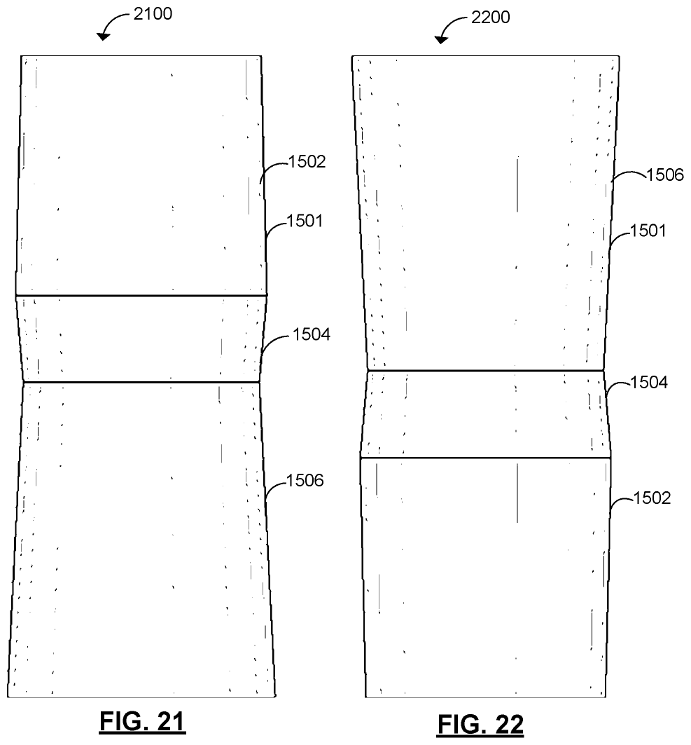

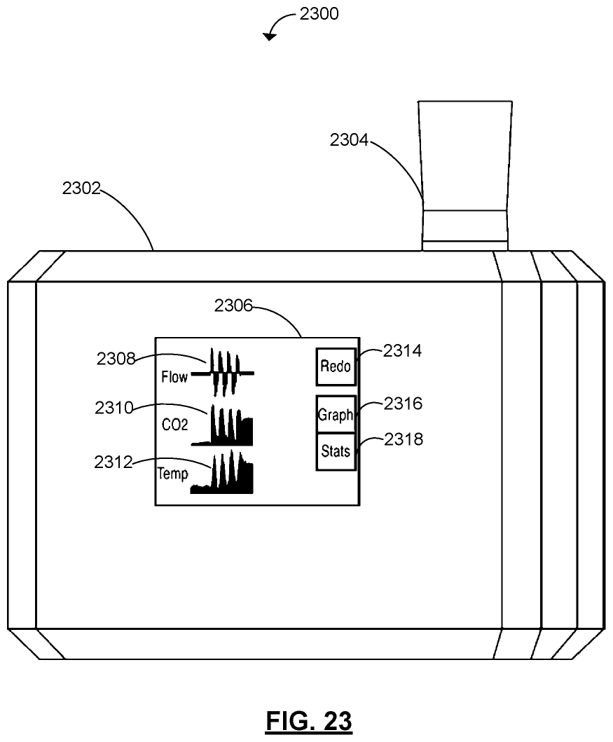

Reference is next made to FIG. 23, showing an example user interface diagram 2300 for a spirometer 2302. A subject may breathe into the mouthpiece 2304 multiple times, during both inhalation and exhalation. The spirometer 2302 records data, including one or more of temperature data, CO₂ concentration data, and air flow data. The spirometer may determine other statistics or generated metrics. The display 2306 may show the collected data, the statistics, or the generated metrics. The display 2306 may show the data in a graph form.

The display may show a flow rate portion 2308, which may include a graph of the collected air flow data.

The display may show a CO₂ concentration portion 2310, which may include a graph of the collected CO₂ concentration portion 2310.

The display may show a temperature portion 2312, which may include a graph of the collected temperature data.

As shown, the data may be collected over multiple inhalation and exhalation cycles.

The display 2306 may be a touch screen, and may include several buttons such as the redo button 2314, graph button 2316, and statistics button 2318.

The redo button 2314 may configure the spirometer to collect another set of data from a subject. The graph button 2316 may trigger the display 2306 to show graphs of the collected data as shown, including the flow rate portion 2308, the CO₂ concentration portion 2310, and the temperature portion 2312.

The statistics button 2318 may trigger the display 2306 to show determined or generated statistics of the flow rate data, CO₂ concentration data, and temperature data.

Reference is next made to FIG. 24, showing system diagram 2400 of the spirometer. The system 2400 has a plurality of a spirometer device 2408, a user device 2410, network 2406, a server 2402, a database 2404 in communication with the server 2402.

Spirometer device 2408 may be a spirometer device as described in embodiments herein.

User device 2410 may be used by an end user to access the data collected from the spirometer 2408 using the data port of the spirometer 2408. The user device 2410 may be connected by a cable to spirometer 2408, or a removable storage device may be used to transfer the data. The data may include CO₂ concentration data, temperature data, and/or air flow data. The user device 2410 may have an application (not shown) running on it to access and/or parse the data from the spirometer 2408. The user device 2410 may transmit the data from the spirometer 2408 to the server 2402 via network 2406, and the server 2402 may store the data in database 2404.

The user device 2410 may be a desktop computer, mobile device, or laptop computer. The user device 2410 may be in network communication with server 2402. The user device 2410 may display the data collected from the spirometer 2408 to a clinician user, or another user. The user at user device 2410 may also be an administrator user who may administer the configuration of the spirometer 2408.

In another embodiment, the user device 2410 may connect to server 2402 and display an application there, and may allow a medical practitioner user to request subject data from prior collected studies of the spirometer.

Network 2406 may be a communication network such as the Internet, a Wide-Area Network (WAN), a Local-Area Network (LAN), or another type of network. Network 104 may include a point-to-point connection, or another communications connection between two nodes.

The server 2402 may be a commercial off-the-shelf device, or a cloud-based server such from Microsoft® Azure® or Amazon® Web Services (AWS®). The server 2402 may have a web server, and/or an Application Programming Interface (API) to collect patient spirometer data.

In another embodiment, the server 2402 may be a medical organization involved in the storage, use, or processing of patient health records. The medical organization may be directly associated with the user device 2410. Alternatively, the server 2402 may be a medical organization indirectly involved with patient health records, for example, user device 2410 may be at a testing facility or a medical clinic not directly associated with server 2402.

The form server 110 is in communication with the database 116, test server 112, and web server 114. A user may access the form server 110 to perform the mapping of candidate forms, and generation of instances of form documents. The form server 110 may automatically map the candidate form. Mapping may involve determining required fields, optional fields, and conditional fields, and associating a key with the field. The key may refer to an element of a structured data set that may be stored in the database 116.

Data from the spirometer 2408 may be sent by user device 2410 in a variety of formats. The formats may include a text-based format such as a file encoded in a markup language such as JavaScript Object Notation (JSON) or eXtensible Markup Language (XML), or another format.

Data received from the server at the user device 2410 may be in a variety of formats, including JavaScript Object Notation (JSON) or eXtensible Markup Language (XML). The server 2402 may generate PDF reports for a subject.

The database 2404 may store subject information, including data recorded from prior spirometry measurement sessions. The database 2404 may be a Structured Query Language (SQL) such as PostgreSQL or MySQL or a not only SQL (NoSQL) database such as MongoDB

Reference is next made to FIG. 25, showing a block diagram 2500 of the spirometer.

The spirometer 2500 has communication unit 2502, display 2504, processor unit 2506, memory unit 2508, I/O unit 2510, user interface 2512, and power unit 2514. The I/O unit 2510 may be in communication with a sensor unit 2516, that may be connected to a temperature sensor 2518, a CO₂ sensor 2520, and a flow sensor 2522.

The temperature sensor 2518 senses the temperature of the subject's breath, and may be an MCP9808 temperature sensor from Microchip®. The temperature sensor 2518 may have a configurable measurement resolution. Since changes in breath temperature are not very large (generally in the range of 0.5-3 degrees Celsius) the temperature sensor may have, for example an accuracy of ±0.0625 degrees, ±0.25° C. (typical) or ±0.5° C. which may be configurable.

The CO₂ sensor 2520 may sense CO₂ concentration directly, or indirectly. For example, the CO₂ concentration may be measured indirectly by the CO₂ sensor 2520 by detecting volatile organic compounds (VOCs). The CO₂ sensor 2520 may itself perform steps to process the raw measurements in order to output a measurement called equivalent CO₂ concentration. The CO₂ sensor 2520 may also measure or determine other VOC concentrations in addition to CO₂ concentration.

The flow rate sensor 2522 may measure the flow rate of the subject's breath through the spirometer. The flow rate sensor may be a SFM330-250-D from Sensirion®. The flow sensor 2522 may have an accuracy of +/−250 standard liters per minute (slm) for human breathing flow measurement. The flow sensor 2522 may be bidirectional, and may function for both a subject's inspiration and expiration through the spirometer. The flow sensor 2522 may be any type of flow sensing technology, including differential pressure sensing or thermal mass flow sensing. Preferably however, thermal mass flow sensing may be used in order to provide accurate sensing for the relatively low volume movement in regular breathing, instead of forced expiratory breathing tests used in other spirometry devices.

The communication unit 2502 may be a standard network adapter such as an Ethernet or 802.11x adapter. The display may be the touchscreen 104 (see FIG. 1) on the outside of the spirometer. The processor unit 2506 may be a standard platform, such an Arduino® or Raspberry Pi®. The processor unit 2506 may be an Advanced RISC Machines (ARM) processor, or another low-power processor.

The processor unit 2506 can also execute a user interface engine 2512 that is used to generate various user interfaces, some examples of which are shown and described herein, such as in FIG. 23. The user interface engine 2512 provides for data collection layouts for users to review collected sensor data, and determined/generated statistics based on the sensor data.

The memory unit 2508 has operating system 2524, programs 2526, a flow sensor measurement engine 2528, a temperature sensor measurement engine 2530, a CO₂ sensor measurement engine 2532, a database 2534, and a real-time clock 2536.

The operating system 2524 may be a Linux-based operating system, an Arduino operating system, or another operating system.

The programs 2526 comprise program code that, when executed, configures the processor unit 2506 to operate in a particular manner to implement various functions and tools for the spirometer.

The flow sensor measurement engine 2528 may perform methods for collecting flow rate data from the flow sensor 2522, and may store the collected data in memory, including at database 2534, at non-volatile storage connected to I/O unit 2516, or both. When the spirometer is powered on, the flow sensor measurement engine 2528 may collect flow sensor calibration data. The flow sensor calibration data may be used to determine a baseline calibration for the flow sensor 2522. This baseline calibration may include an offset value.

The flow sensor measurement engine 2528 may collect a plurality of flow sensor readings over a time-series from the flow sensor 2522. The plurality of flow sensor measurements may be taken in addition to other sensor readings over a time-series. The flow sensor measurement engine 2528 may also determine statistics and determined/generated data from the plurality of flow sensor readings. This may include averages, means, maximums, minimums, rates of change (including first and second derivatives) of the plurality of flow sensor measurements. The determine statistics and determined/generated data may be stored in database 2534, and/or non-volatile storage connected via I/O Unit 2510.

The CO₂ sensor measurement engine 2532 may perform methods for collecting carbon dioxide measurements from the CO₂ sensor 2520, and may store the collected data in memory, including at database 2534, at non-volatile storage connected to I/O unit 2516, or both. When the spirometer is powered on, the CO₂ sensor measurement engine 2532 may collect CO₂ sensor calibration data. The CO₂ sensor calibration data may be used to determine a baseline calibration for the CO₂ sensor 2520. This baseline calibration may include an offset value.

The CO₂ sensor measurement engine 2532 may collect a plurality of CO₂ sensor readings over a time-series from the CO₂ sensor 2520. The plurality of CO₂ sensor measurements may be taken in addition to other sensor readings over a time-series. The CO₂ sensor measurement engine 2532 may also determine statistics and determined/generated data from the plurality of CO₂ sensor readings. This may include averages, means, maximums, minimums, rates of change (including first and second derivatives) of the plurality of CO₂ sensor measurements. The determine statistics and determined/generated data may be stored in database 2534, and/or non-volatile storage connected via I/O Unit 2510.

The temperature sensor measurement engine 2530 may perform methods for collecting temperature data from the temperature sensor 2518, and may store the collected data in memory, including at database 2534, at non-volatile storage connected to I/O unit 2516, or both. When the spirometer is powered on, the temperature sensor measurement engine 2530 may collect temperature sensor calibration data. The temperature sensor calibration data may be used to determine a baseline calibration for the temperature sensor 2518. This baseline calibration may include an offset value.

The temperature sensor measurement engine 2530 may collect a plurality of temperature sensor readings over a time-series from the temperature sensor 2518. The plurality of temperature sensor measurements may be taken in addition to other sensor readings over a time-series. The temperature sensor measurement engine 2530 may also determine statistics and determined/generated data from the plurality of temperature sensor readings. This may include averages, means, maximums, minimums, rates of change (including first and second derivatives) of the plurality of temperature sensor measurements. The determine statistics and determined/generated data may be stored in database 2534, and/or non-volatile storage connected via I/O Unit 2510.

The temperature sensor measurement engine 2530, flow sensor measurement engine 2528, and CO₂ sensor measurement engine 2532 may individually or collectively perform zero-crossing measurements based on data from the temperature sensor 2518, flow sensor 2522, and CO₂ sensor 2520 respectively.

The zero crossing measurement may be done in order to count the number of breaths since the start of data collection. This may be used if the spirometer is used for a study where the study protocol dictates the collection of breathing data for a specific number of breaths or a minimum number of breaths, etc.

The temperature sensor measurement engine 2530, flow sensor measurement engine 2528, and CO₂ sensor measurement engine 2532 may collect data at the same frequency, for example, 2 Hz, 3 Hz, 4 Hz, 5 Hz or 10 Hz for the data collection period of at least 5, 10, 15, 20 or 30 seconds, and optionally at least 2, 3, 4, 5 subject breaths. The data collection frequency may be selected based on the slowest sensor of the temperature sensor 2518, the CO₂ sensor 2520, and the flow sensor 2522.

Alternatively, the temperature sensor measurement engine 2530, flow sensor measurement engine 2528, and CO₂ sensor measurement engine 2532 may collect data at different frequencies based on the sampling speed of each of the temperature sensor 2518, the CO₂ sensor 2520, and the flow sensor 2522.

The database 2534 may be an SQL database as known, such as a SQLlite® database, or another database.

The real-time clock 2536 may be provided in order to synchronize the collection of data from the one or more sensors. The real-time clock 2536 may be internal or external to the processor and/or processor logic board. The real-time clock 2536 may be a PCF8523 from NXP®. The spirometer uses the real-time clock 2536 to storage collected sensor measurements with an accurate date and time. The real-time clock may be accurate up to 2 seconds a day. The real-time clock may be an additional clock beyond an internal processor clock that may be used in order to synchronize the data collection.

I/O unit 2510 provides access to storage devices including disks and memory cards. The I/O unit 2510 can include a connection to a non-volatile storage card such as an SD card. The I/O unit 2510 may be a wired connection, such as a USB connection. The I/O unit provides local storage access to the software running on the spirometer so that collected sensor data and generated/determined metrics can be written to the non-volatile storage.

The power unit 216 provides power to the spirometer, via a battery means or by a powered connection such as a USB cable.

Reference is next made to FIG. 26, showing a method diagram 2600 of the spirometer.

Optionally, at 2602, the method comprises collecting, at a processor, a plurality of baseline sensor values.

Optionally, at 2604, the method comprises determining, at the processor, a plurality of calibration values from the plurality of baseline sensor values.

At 2606, the method comprises receiving, at a processor, a plurality of measured sensor values. The plurality of measured sensor values may be from one or more sensors in communication with the processor. The one or more sensors may include a flow rate sensor, a temperature sensor, and a CO₂ sensor. The plurality of measured sensor values may include a plurality of CO₂ concentration values of the breath sensed by a CO₂ sensor, a plurality of temperature values sensed by a temperature sensor; and a plurality of flow rate values sensed by a flow rate sensor for sensing of the breath.

The receiving, at the processor, the plurality of measured sensor values may occur at a predetermined frequency over a data collection period; and the plurality of measured sensor values and the plurality of output sensor values may be time series data.

The predetermined frequency may be at least 2, Hz, 3 Hz, 4 Hz, 5 Hz or 10 Hz and the data collection period may be at least 5, 10, 15, 20 or 30 seconds, optionally at least 2, 3, 4, 5 subject breaths.

There may be more than a single predetermined frequency, for example, each sensor may have a pre-determined frequency.

At 2608, the method comprises determining, at the processor, a plurality of output values from a plurality of calibration values and the plurality of measured sensor values. The processor may further determine statistics, or generated/determined values that may also be included in the output values, including but not limited to:
A plurality of first determined temperature values based on the plurality of output sensor values, where the plurality of first determined temperature values are a function of instantaneous air flow. This may be a first derivative of temperature values;
One or more inflection points of the plurality of first determined temperature values;
A plurality of second determined temperature values based on the plurality of first determined temperature values, wherein the second determined temperature values are a function of percentage of air exhaled. This may be a second derivative of temperature values;
One or more inflection points of the plurality of second determined temperature values;
A plurality of first determined CO₂ concentration values, based on the plurality of output sensor values, wherein the plurality of first determined CO₂ concentration values are a function of instantaneous air flow. This may be a first derivative of CO₂ concentration values;
One or more inflection points of the plurality of first determined CO₂ concentration values;
A plurality of second determined CO₂ concentration values based on the plurality of first determined temperature values, wherein the second determined CO₂ concentration values are a function of percentage of air exhaled. This may be a second derivative of CO₂ concentration values.; and/or
One or more inflection points of the plurality of second determined CO₂ concentration values.

At 2610, the method comprises storing, at a memory in communication with the processor, the plurality of output values.

Reference is next made to FIG. 27, showing an example output diagram 2700 of the spirometer. The output of the spirometer may be provided via the touchscreen of the spirometer, or may be downloaded to a user device from the spirometer using the data port. As shown, the downloaded data from the spirometer may include CO₂ concentration, temperature, and flow rate over a time series for several subject breaths.

In one embodiment, the spirometer and/or systems described herein may be used for determining a diagnosis or prognosis of breathing difficulties and/or lung function for a subject. Also provided are systems configured for determining a diagnosis or prognosis of breathing difficulties and/or lung function for a subject according to a method described herein.

For example, in one embodiment, there is provided a method for determining a diagnosis or prognosis for a subject comprising providing a spirometer as described herein and obtaining CO₂ concentration values, temperature and/or flow rates of air in the flow path for the subject breathing through the flow path. In one embodiment, the method comprises generating one or more output values based on the CO₂ concentration values, temperature and/or flow rates and comparing the one or more output values to one or more control values. In one embodiment, the control values are representative of subjects with predetermined status with respect to breathing difficulties or lung function. In one embodiment, the output values and/or the control values are based on a time series of CO₂ concentration values, temperature and/or flow rates.

For example, in one embodiment the output value is a change in exhaled air temperature (Δe° T) and the method is for determining a diagnosis or prognosis of a subject with or suspected of having chronic obstructive pulmonary disease. Paredi et al. European Respiratory Journal 2003 21: 439-443 (2003), report that exhaled temperature increase is reduced in chronic obstructive pulmonary disease patients compared to normal controls.

In another embodiment, the output value is a change of exhaled breath temperature and the method is for determining a diagnosis or prognosis of a subject with or suspected of having asthma. Paredi et al. Am J Respir Crit Care Med. 2002 Jan. 15; 165(2):181-4, describes that Δe° T was higher in asthmatic patients compared with normal subjects.

All publications, patents and patent applications and are herein incorporated by reference in their entirety to the same extent as if each individual publication, patent, or patent application was specifically and individually indicated to be incorporated by reference in its entirety.

The present invention has been described here by way of example only. Various modification and variations may be made to these exemplary embodiments without departing from the spirit and scope of the invention, which is limited only by the appended claims.

### EXAMPLES

### Example 1: Spirometer Performance Study

A study was performed to first investigate the performance of the spirometer results to an asthma control, and second to compare the performance results to asthma symptom exacerbations (i.e. worsening asthma symptoms).

### Study Design and Participants

The study included 16 individuals with diagnosed asthma. Two of these individuals had uncontrolled asthma (defined as an Asthma Control Test score of less than 20), and remaining study members had controlled asthma.

There were nine experiments performed on each participant. Participants performed the experiments in a temperature and humidity controlled room. The participant first performed the experiments at 20 degrees Celsius. Spirometry and forced oscillation technique was performed at the beginning of the experiment, then repeated after 10 minutes of rest (seated), then repeated a third time after exercise. These three experiments were then repeated for 0 degrees Celsius, then at 10 degrees Celsius. Patient symptom reports were received for all individuals for all the experiments.

With the available information, two branches of analysis could be performed on asthma patients:
1) compare spirometer results to asthma control; and
2) compare spirometer results to asthma symptom exacerbations (i.e. worsening asthma symptoms).

Furthermore, the study included two healthy participants. The results from these healthy individuals can be compared to all asthmatic individuals, as well as asthmatic individuals who were symptomatic.

### Results

TABLE 1a
Uncontrolled Asthma Group vs Controlled Asthma Group

| | | Second | Time | Time | CO2 | | |
|---|---|---|---|---|---|---|---|
| | | Breath | to | to | Increase − | | Area Flow |
| | | Max | Temp | CO2 | Temp | Breathing | Before |
| | | Temp | Increase | Increase | Increase | Rate | CO2 Rise |
| controlled | average | 27.06469 | 536.6491 | 770.2632 | 233.614 | 18.12325 | 0.196106 |
| | stdv | 1.187396 | 210.2779 | 268.4131 | 323.5535 | 10.10236 | 0.07913 |
| uncontrolled | average | 26.51389 | 451.1111 | 643.6111 | 192.5 | 18.97056 | 0.180563 |
| | stdv | 0.742473 | 193.1659 | 186.6094 | 196.4129 | 4.58012 | 0.053683 |
| | pval | 0.005395 | 0.104718 | 0.008999 | 0.304677 | 0.182382 | 0.208814 |

TABLE 1b
Uncontrolled Asthma Group vs Controlled Asthma Group

| | | | | | Area | | |
|---|---|---|---|---|---|---|---|
| | | Area | Area | Area | After | | |
| | | Flow | Flow | Flow | CO2 * | | |
| | | After | Before | After | Area | Inhale/ | |
| | | CO2 | Temp | Temp | After | Exhale | Exhale |
| | | Rise | Rise | Rise | Temp | Switch | Propotion |
| controlled | average | 0.349302 | 0.125593 | 0.421929 | 0.206368 | 0.427789 | 0.553692 |
| | stdv | 0.230753 | 0.088453 | 0.272978 | 0.256777 | 0.054292 | 0.041673 |
| uncontrolled | average | 0.188298 | 0.120637 | 0.246489 | 0.060276 | 0.34773 | 0.645782 |
| | stdv | 0.116247 | 0.073627 | 0.143189 | 0.071723 | 0.099348 | 0.04512 |
| | pval | 3.82E−06 | 0.872528 | 1.87E−05 | 5.63E−07 | 0.003851 | 5.89E−08 |

TABLE 1c
Uncontrolled Asthma Group vs Controlled Asthma Group

| | | V1/ | V1/ | V2/ | T1/ | T1/ | T2/ |
|---|---|---|---|---|---|---|---|
| | | V2 | Vtotal | Vtotal | T2 | Ttotal | Ttotal |
| controlled | average | 1.071712 | 0.408309 | 0.591691 | 0.579547 | 0.286007 | 0.713993 |
| | stdv | 1.760045 | 0.180053 | 0.180053 | 0.777609 | 0.21667 | 0.21667 |
| uncontrolled | average | 1.21592 | 0.508784 | 0.491216 | 0.710748 | 0.334662 | 0.665338 |
| | stdv | 0.672326 | 0.144563 | 0.144563 | 0.695835 | 0.223055 | 0.223055 |
| | pval | 0.163379 | 0.002413 | 0.002413 | 0.089376 | 0.153998 | 0.153998 |

TABLE 2
Proportion of time exhaling (20 Celsius, resting)

| ID | Asthma | Proportion |
|---|---|---|
| KMV01 | Mild-Moderate | 0.510204082 |
| KMV02 | Mild-Moderate | 0.575809199 |
| KMV03 | Mild-Moderate | 0.529411765 |
| KMV04 | Mild-Moderate | 0.566484517 |
| KMV05 | Mild-Moderate | 0.511784512 |
| KMV06 | Mild-Moderate | 0.53422619 |
| KMV07 | Mild-Moderate | 0.600870827 |
| KMV08 | Severe | 0.618887015 |
| KMV09 | Mild-Moderate | 0.517503805 |
| KMV10 | Severe | 0.617777778 |
| KMV11 | Mild-Moderate | 0.607876712 |
| KMV12 | Mild-Moderate | |
| KMV13 | Mild-Moderate | 0.555369128 |
| KMV14 | Moderate | 0.543293718 |
| KMV15 | Mild-Moderate | 0.555732484 |
| KMV16 | Moderate | 0.577054795 |
| | Average | Stdv |
| Controlled | 0.552740133 | 0.032131573 |
| Uncontrolled | 0.618332396 | 0.000784349 |
| pval | 8.52591E−06 | |

TABLE 3
CO₂ Rate of Change (20 Celsius, resting)

| ID | Asthma | ROC |
|---|---|---|
| KMV01 | Mild-Moderate | 1.324 |
| KMV02 | Moderate | 1.418 |
| KMV03 | Mild-Moderate | 1.407 |
| KMV04 | Moderate | 1.541 |
| KMV05 | Moderate | 2.103 |
| KMV06 | Mild-Moderate | 1.211 |
| KMV07 | Mild-Moderate | 0.882 |
| KMV08 | Severe | 0.901 |
| KMV09 | Moderate | 1.412 |
| KMV10 | Severe | 0.99 |
| KMV11 | Mild-Moderate | 1.539 |
| KMV12 | Moderate | |
| KMV13 | Moderate | 1.696 |
| KMV14 | Moderate | 1.2486 |
| KMV15 | Moderate | 1.68259 |
| KMV16 | Moderate-Severe | 1.3937 |
| | Average | Stdv |
| Controlled | 1.426333 | 0.323556 |
| Uncontrolled | 0.9455 | 0.062933 |
| pval | 0.00266 | |

TABLE 4
CO₂ Rate of Change (0 Celsius, exercising)

| ID | Asthma | ROC |
|---|---|---|
| KMV01 | Mild-Moderate | 1.301 |
| KMV02 | Moderate | 1.913 |
| KMV03 | Mild-Moderate | 1.271 |
| KMV04 | Moderate | 1.294 |
| KMV05 | Moderate | 1.261 |
| KMV06 | Mild-Moderate | 1.181 |
| KMV07 | Mild-Moderate | 0.911 |
| KMV08 | Severe | 0.837 |
| KMV09 | Moderate | 1.626 |
| KMV10 | Severe | 0.803 |
| KMV11 | Mild-Moderate | 1.066 |
| KMV12 | Moderate | |
| KMV13 | Moderate | 1.576581 |
| KMV14 | Moderate | 1.40472 |
| KMV15 | Moderate | 1.046618 |
| KMV16 | Moderate-Severe | 1.442374 |
| | Average | Stdv |
| Controlled | 1.313778 | 0.296432 |
| Uncontrolled | 0.82 | 0.024042 |
| pval | 0.000996 | |

TABLE 5a
Symptomatic Group vs. Asymptomatic Group

| | | Second | Time | Time | CO2 | | Area |
|---|---|---|---|---|---|---|---|
| | | Breath | to | to | Increase − | | Flow |
| | | Max | Temp | CO2 | Temp | Breathing | Before |
| | | Temp | Increase | Increase | Increase | Rate | CO2 Rise |
| asymptomatic | average | 27.16842 | 548 | 784.7474 | 236.7474 | 17.56989 | 0.195121 |
| | stdv | 1.160602 | 218.5996 | 285.1017 | 341.5605 | 10.7336 | 0.083577 |
| symptomatic | average | 26.53041 | 465.8919 | 671.4595 | 205.5676 | 19.95622 | 0.191074 |
| | stdv | 0.999308 | 172.6567 | 166.6791 | 205.4439 | 5.052255 | 0.053555 |
| | p-val | 0.000594 | 0.031441 | 0.00155 | 0.297862 | 0.007246 | 0.516922 |

TABLE 5b
Symptomatic Group vs. Asymptomatic Group

| | | | | | Area | | |
|---|---|---|---|---|---|---|---|
| | | Area | Area | Area | After | | |
| | | Flow | Flow | Flow | CO2 * | | |
| | | After | Before | After | Area | Inhale/ | |
| | | CO2 | Temp | Temp | After | Exhale | Exhale |
| | | Rise | Rise | Rise | Temp | Switch | Propotion |
| asymptomatic | average | 0.365996 | 0.126904 | 0.436518 | 0.225305 | 0.432046 | 0.549548 |
| | stdv | 0.242956 | 0.091408 | 0.287469 | 0.274532 | 0.053554 | 0.040795 |
| symptomatic | average | 0.228113 | 0.119817 | 0.299121 | 0.086675 | 0.377911 | 0.609134 |
| | stdv | 0.128539 | 0.072627 | 0.164719 | 0.087159 | 0.083812 | 0.055701 |
| | p-val | 1E−06 | 0.749955 | 2.69E−05 | 3.78E−06 | 0.000827 | 5.44E−07 |

TABLE 5c
Symptomatic Group vs. Asymptomatic Group

| | | V1/ | V1/ | V2/ | T1/ | T1/ | T2/ |
|---|---|---|---|---|---|---|---|
| | | V2 | Vtotal | Vtotal | T2 | Ttotal | Ttotal |
| asymptomatic | average | 1.0646 | 0.398094 | 0.601906 | 0.591989 | 0.286062 | 0.713938 |
| | stdv | 1.87485 | 0.183877 | 0.183877 | 0.827019 | 0.221908 | 0.221908 |
| symptomatic | average | 1.160127 | 0.483418 | 0.516582 | 0.611764 | 0.309533 | 0.690467 |
| | stdv | 0.87234 | 0.149395 | 0.149395 | 0.59169 | 0.207095 | 0.207095 |
| | p-val | 0.195363 | 0.00017 | 0.00017 | 0.03558 | 0.107892 | 0.107892 |

TABLE 6a
Healthy Group vs. Asthma Group

| | | Second | Time | Time | Prop | Prop | CO2 |
|---|---|---|---|---|---|---|---|
| | | Breath | to | to | Time to | Time to | Increase − |
| | | Max | Temp | CO2 | Temp | CO2 | Temp |
| | | Temp | Increase | Increase | Increase | Increase | Increase |
| Healthy | Average | 27.25 | 572.8333 | 849.2222 | 0.140024 | 0.195298 | 276.3889 |
| | stdv | 1.104802 | 249.5052 | 266.4727 | 0.081677 | 0.063588 | 279.7161 |
| Asthma | Average | 26.98958 | 524.9848 | 752.9924 | 0.155396 | 0.205864 | 228.0076 |
| | stdv | 1.150531 | 209.4077 | 261.8563 | 0.081552 | 0.063689 | 309.0458 |
| | pval | 0.360591 | 0.445986 | 0.164061 | 0.461698 | 0.515393 | 0.503616 |

TABLE 6b
Healthy Group vs. Asthma Group

| | | | Area | Area | Area | Area | |
|---|---|---|---|---|---|---|---|
| | | | Flow | Flow | Flow | Flow | |
| | | | Before | After | Before | After | Inhale/ |
| | | Breathing | CO2 | CO2 | Temp | Temp | Exhale |
| | | Rate | Rise | Rise | Rise | Rise | Switch |
| Healthy | Average | 14.72167 | 0.184574 | 0.436505 | 0.104125 | 0.518264 | 0.411412 |
| | stdv | 4.790679 | 0.073077 | 0.268247 | 0.069385 | 0.325541 | 0.030949 |
| Asthma | Average | 18.23879 | 0.193986 | 0.327347 | 0.124917 | 0.398005 | 0.416872 |
| | stdv | 9.531106 | 0.076183 | 0.225301 | 0.086344 | 0.26569 | 0.067706 |
| | pval | 0.016345 | 0.615064 | 0.114465 | 0.259004 | 0.148918 | 0.563433 |

TABLE 6c
Healthy Group vs. Asthma Group

| | | Exhale | V1/ | V1/ | V2/ | T1/ | T1/ | T2/ |
|---|---|---|---|---|---|---|---|---|
| | | Propotion | V2 | Vtotal | Vtotal | T2 | Ttotal | Ttotal |
| Healthy | Average | 0.593575 | 0.598959 | 0.338751 | 0.661249 | 0.381015 | 0.215833 | 0.784167 |
| | stdv | 0.089843 | 0.444882 | 0.145009 | 0.145009 | 0.476813 | 0.189092 | 0.189092 |
| Asthma | Average | 0.56625 | 1.091377 | 0.42201 | 0.57799 | 0.597574 | 0.292641 | 0.707359 |
| | stdv | 0.052617 | 1.653251 | 0.178534 | 0.178534 | 0.765718 | 0.217331 | 0.217331 |
| | pval | 0.222796 | 0.006805 | 0.036054 | 0.036054 | 0.107965 | 0.125976 | 0.125976 |

TABLE 7a
Healthy Group vs. Symptomatic Asthma Group

| | | Second | Time | Time | Prop | Prop | CO2 |
|---|---|---|---|---|---|---|---|
| | | Breath | to | to | Time to | Time to | Increase − |
| | | Max | Temp | CO2 | Temp | CO2 | Temp |
| | | Temp | Increase | Increase | Increase | Increase | Increase |
| Healthy | Average | 27.25 | 572.8333 | 849.2222 | 0.140024 | 0.195298 | 276.3889 |
| | stdv | 1.104802 | 249.5052 | 266.4727 | 0.081677 | 0.063588 | 279.7161 |
| Symptomatic | Average | 26.53041 | 465.8919 | 671.4595 | 0.156191 | 0.217268 | 205.5676 |
| Asthma | stdv | 0.999308 | 172.6567 | 166.6791 | 0.072142 | 0.062329 | 205.4439 |
| | pval | 0.026086 | 0.113919 | 0.016009 | 0.480078 | 0.234797 | 0.347775 |

TABLE 7b
Healthy Group vs. Symptomatic Asthma Group

| | | | Area | Area | Area | Area | |
|---|---|---|---|---|---|---|---|
| | | | Flow | Flow | Flow | Flow | |
| | | | Before | After | Before | After | Inhale/ |
| | | Breathing | CO2 | CO2 | Temp | Temp | Exhale |
| | | Rate | Rise | Rise | Rise | Rise | Switch |
| Healthy | Average | 14.72167 | 0.184574 | 0.436505 | 0.104125 | 0.518264 | 0.411412 |
| | stdv | 4.790679 | 0.073077 | 0.268247 | 0.069385 | 0.325541 | 0.030949 |
| Symptomatic | Average | 19.95622 | 0.191074 | 0.228113 | 0.119817 | 0.299121 | 0.377911 |
| Asthma | stdv | 5.052255 | 0.053555 | 0.128539 | 0.072627 | 0.164719 | 0.083812 |
| | pval | 0.000659 | 0.739541 | 0.00513 | 0.443543 | 0.013495 | 0.036458 |

TABLE 7c
Healthy Group vs. Symptomatic Asthma Group

| | | Exhale | V1/ | V1/ | V2/ | T1/ | T1/ | T2/ |
|---|---|---|---|---|---|---|---|---|
| | | Propotion | V2 | Vtotal | Vtotal | T2 | Ttotal | Ttotal |
| Healthy | Average | 0.593575 | 0.598959 | 0.338751 | 0.661249 | 0.381015 | 0.215833 | 0.784167 |
| | stdv | 0.089843 | 0.444882 | 0.145009 | 0.145009 | 0.476813 | 0.189092 | 0.189092 |
| Symptomatic | Average | 0.609134 | 1.160127 | 0.483418 | 0.516582 | 0.611764 | 0.309533 | 0.690467 |
| Asthma | stdv | 0.055701 | 0.87234 | 0.149395 | 0.149395 | 0.59169 | 0.207095 | 0.207095 |
| | pval | 0.506636 | 0.002621 | 0.001543 | 0.001543 | 0.128221 | 0.103289 | 0.103289 |

### Analysis 1: Controlled vs Uncontrolled Asthma

### Every Experiment

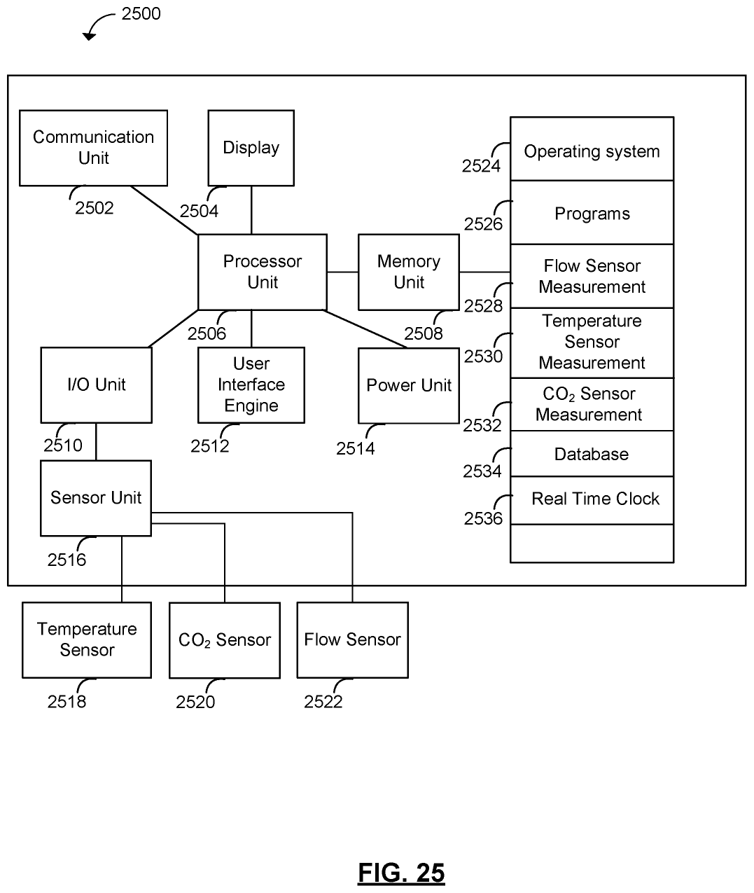

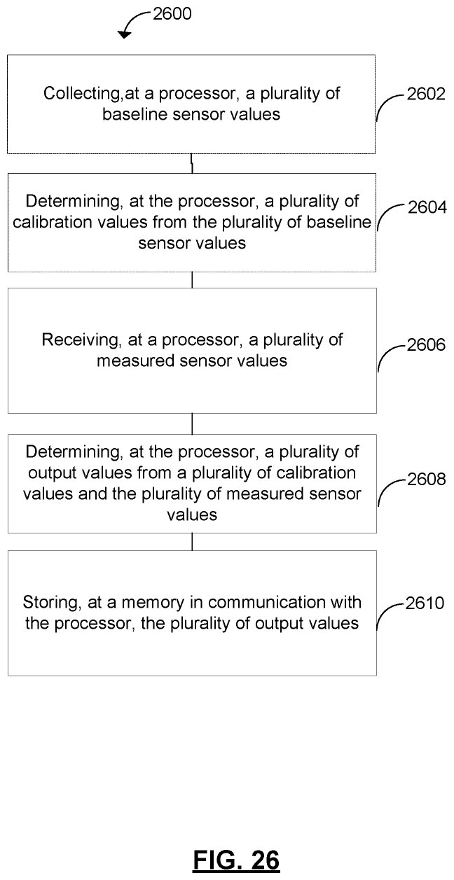

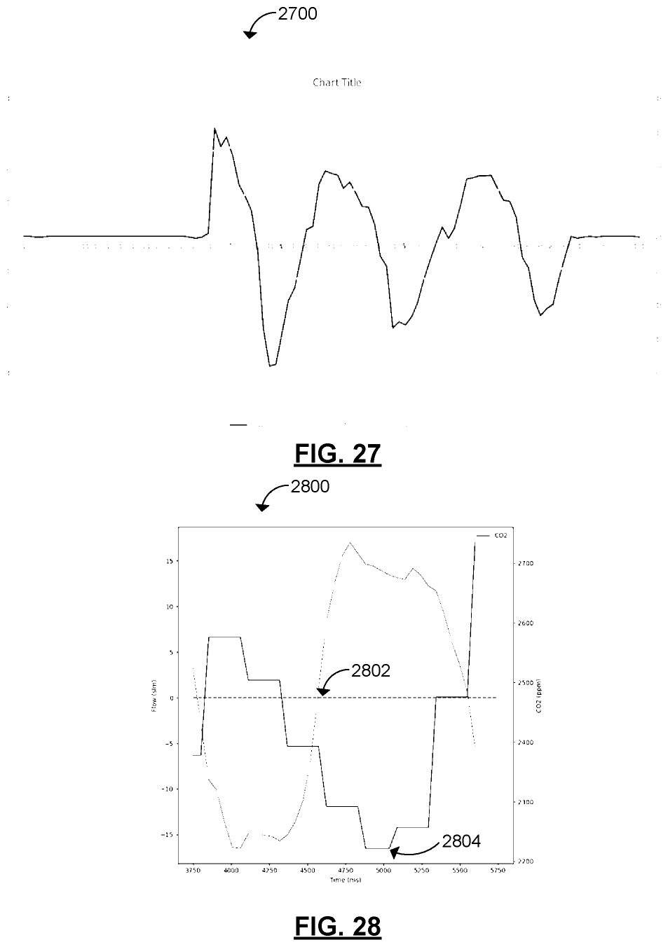

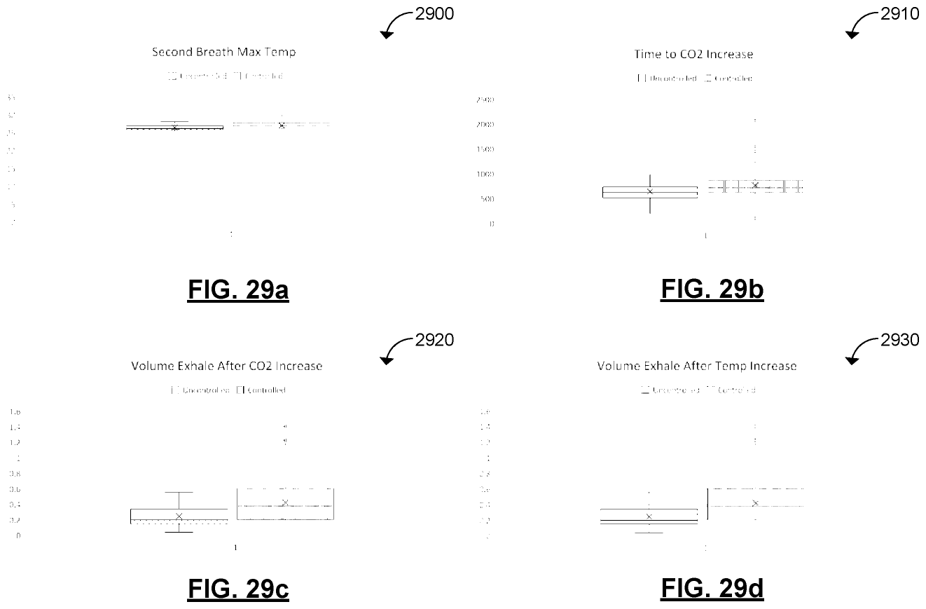

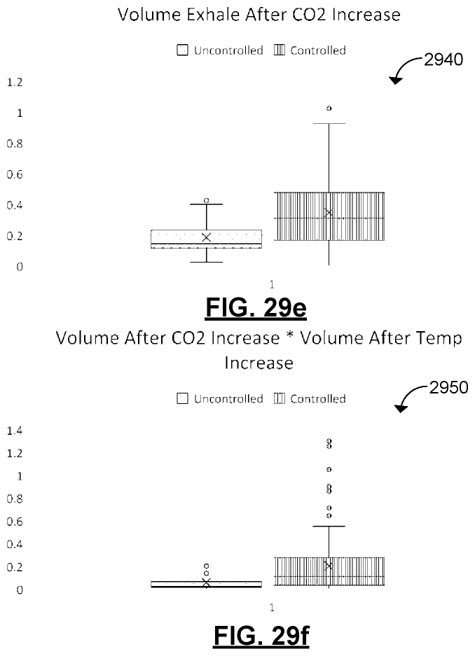

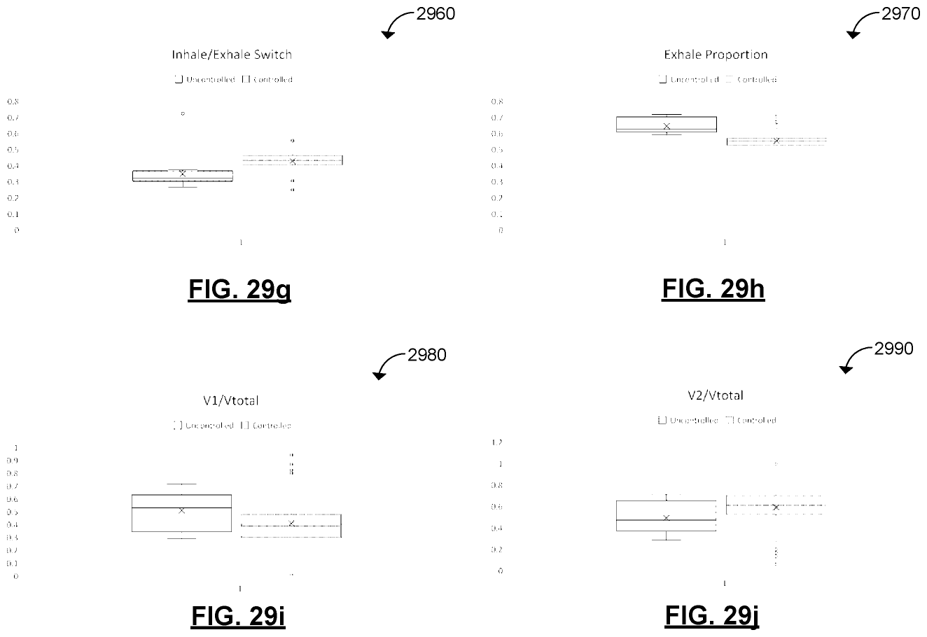

Each experiment was labeled as either controlled or uncontrolled based on participants' ACT score (<20 uncontrolled). Mean and standard deviation of each feature for all experiments was calculated, and statistical significance was determined for the mean of the two groups. Results can be seen in Tables 1a, 1b and 1c above, and FIGS. 29a, 29b, 29c, 29d, 29e, 29f, 29g, 29h, 29i and 29j.

Significant Features include: Single Breath Max Temp, Time to CO₂ Increase, Area Flow After CO₂ Rise, Area Flow after Temperature Rise, Area After CO₂ Rise*Area After Temp Rise, Inhale-Exhale Switch, Exhale Proportion, V1/Vtotal, V2/Vtotal

### Select Experiments

Additional analysis was performed, comparing individuals with controlled and uncontrolled asthma within the same experiment. Previous literature has indicated that asthmatic individuals spend more time per breath exhaling than their non-asthmatic counterparts. Two results stem from this notion and can be interpreted from the spirometer data:

1. Overall proportion of time exhaling over entire time series

2. Average rate of change of top CO2 increases and decreases

Results can be seen in Tables 2, 3 and 4 above.

### Analysis 2: Asthma Symptoms

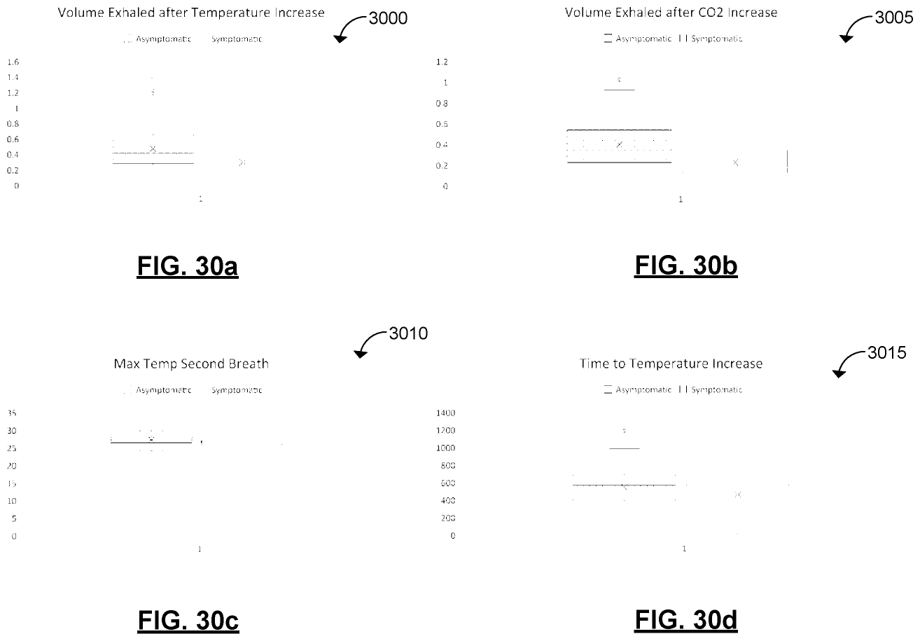

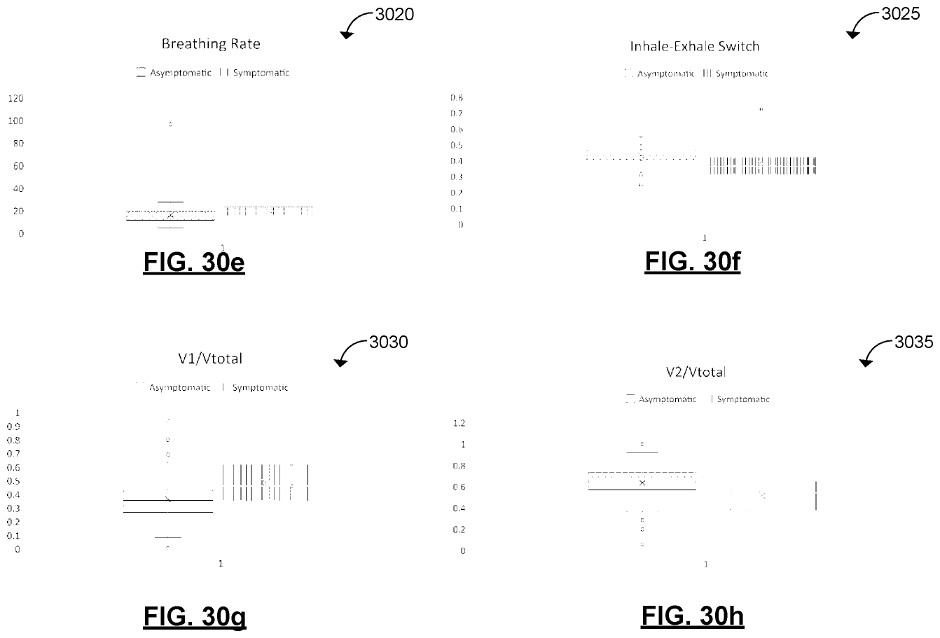

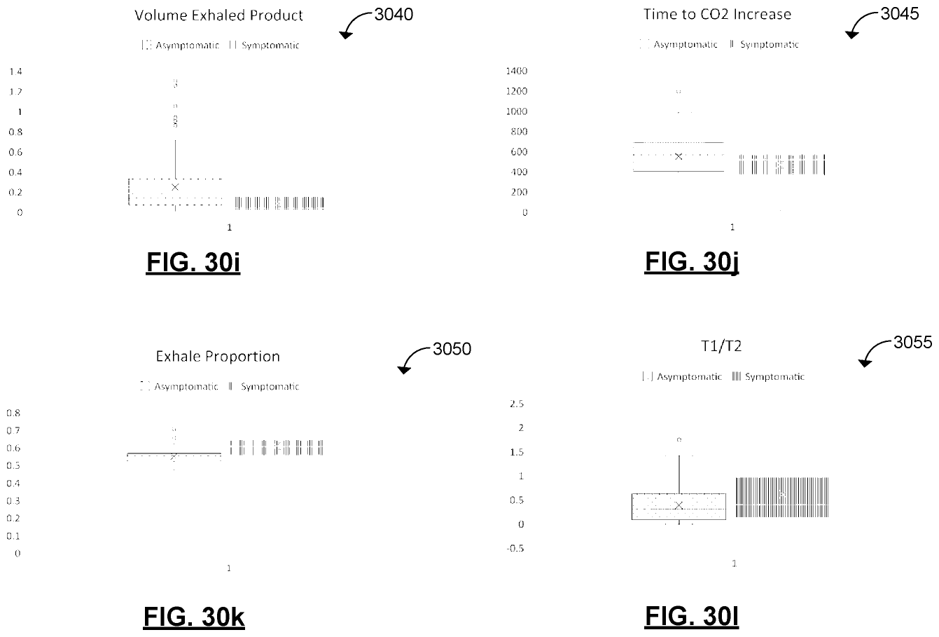

The average of the three symptom ratings (shortness of breath, dyspnea, chest pain; rated on a scale from 0-5) was taken for each experiment for each individual. Individuals were grouped as symptomatic (average symptom rating >=1) or asymptomatic (average symptom rating <1). Mean and standard deviation of each feature for all experiments was calculated, and statistical significance was determined for the mean of the two groups. Results can be seen in Tables 5a, 5b and 5c (above) and FIGS. 30a, 30b, 30c, 30d, 30e, 30f, 30g, 30h, 30i, 30j, 30k, and 30l.

Significant Features include: Selected Breath Max Temp, Time to Temp Increase, Time to CO2 Increase, Breathing Rate, Area Flow After CO2 Rise, Area Flow after Temperature Rise, Area After CO2 Rise*Area After Temp Rise, Inhale/Exhale Switch, Exhale Proportion, V1/Vtotal, V2/Vtotal, T1/T2.

### Analysis 3: Healthy vs Asthma

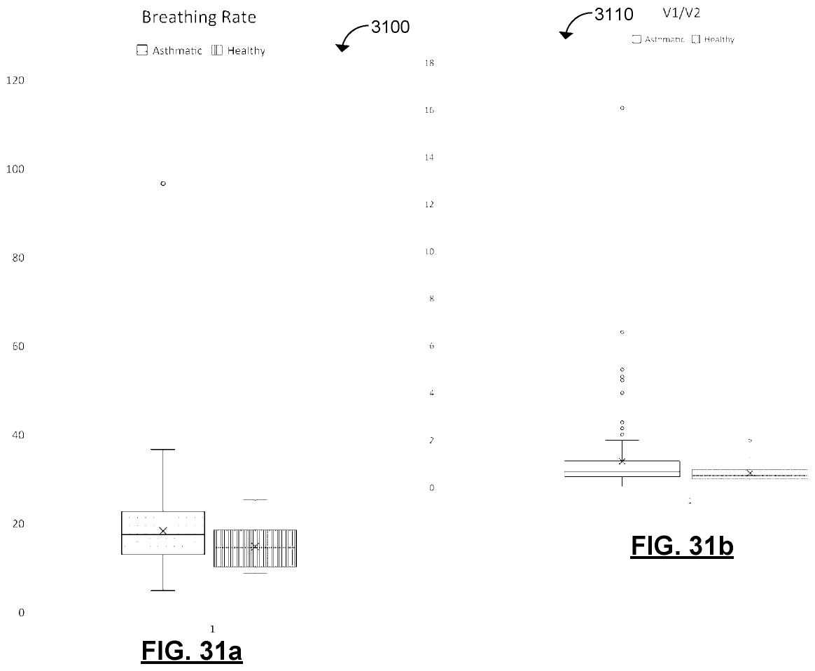

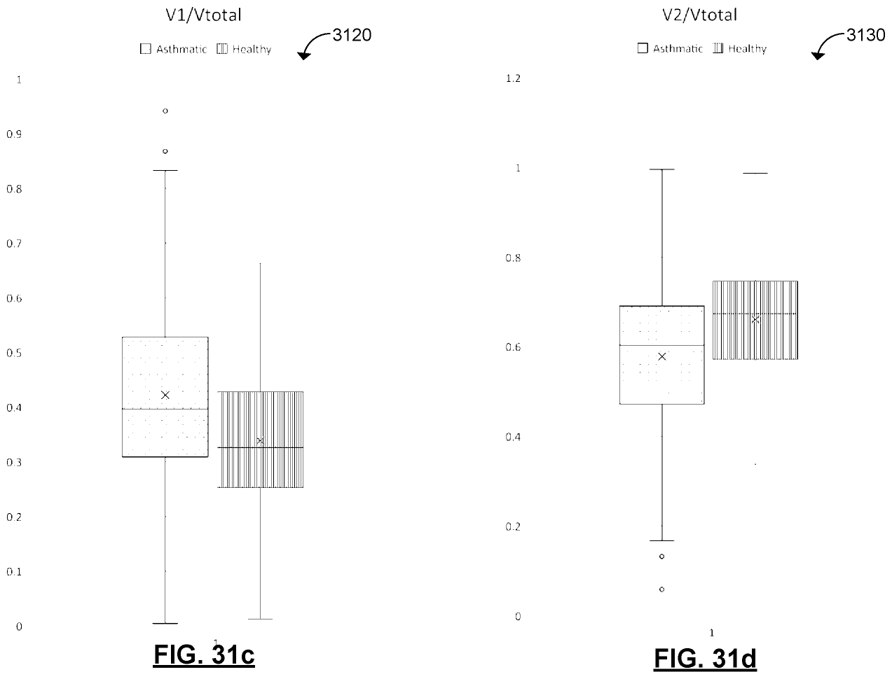

All experiments conducted by healthy individuals were compared to all experiments performed by asthmatic individuals. Mean and standard deviation of each feature for all experiments was calculated, and statistical significance was determined for the mean of the two groups. Results can be seen in Tables 6a, 6b and 6c (above) and FIGS. 31a, 31b, 31c and 31d.

Significant features include: Breathing rate, V1/V2, V1/Vtotal, V2/Vtotal

### Analysis 4: Healthy vs Symptomatic Asthma

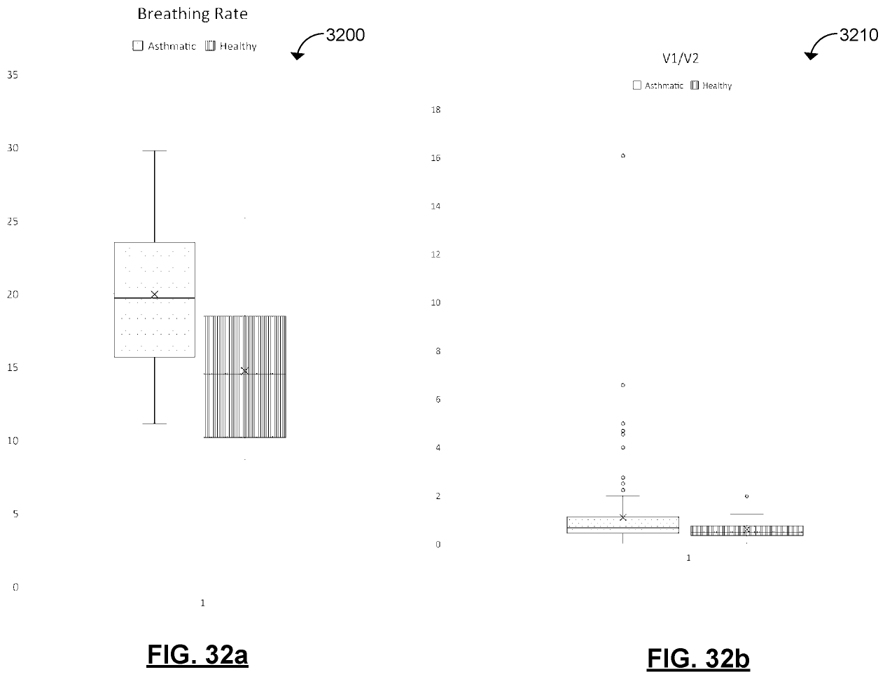

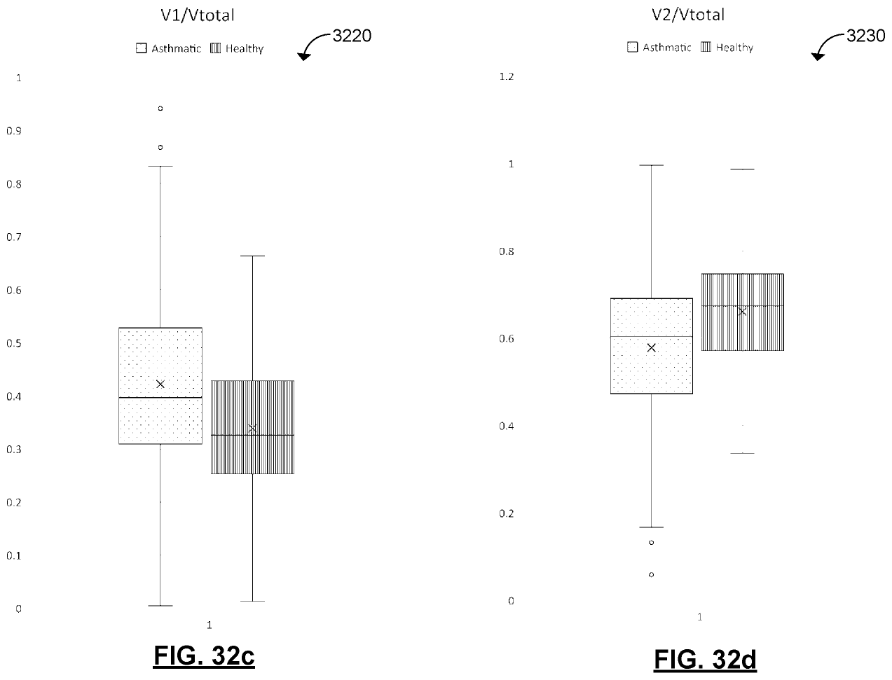

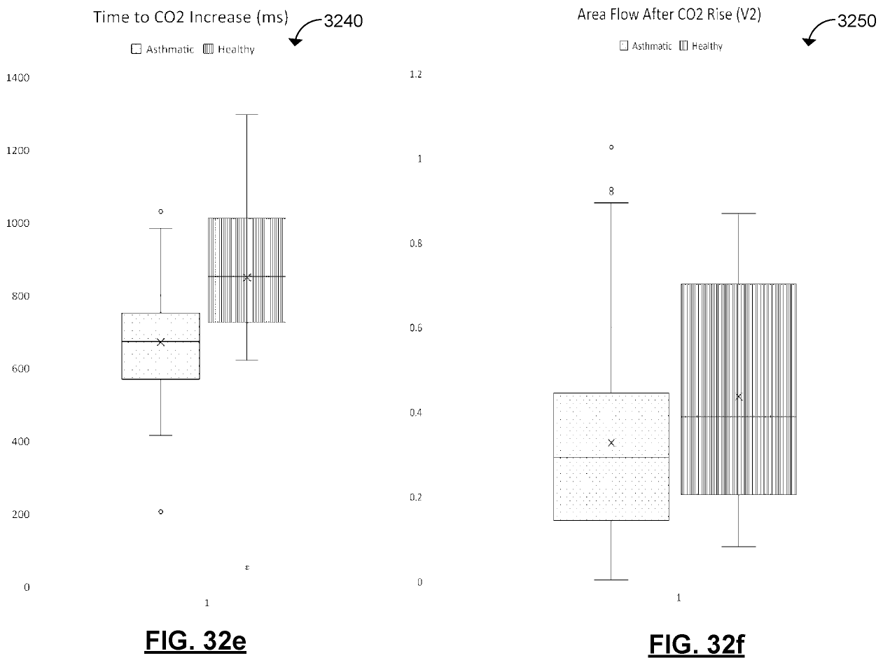

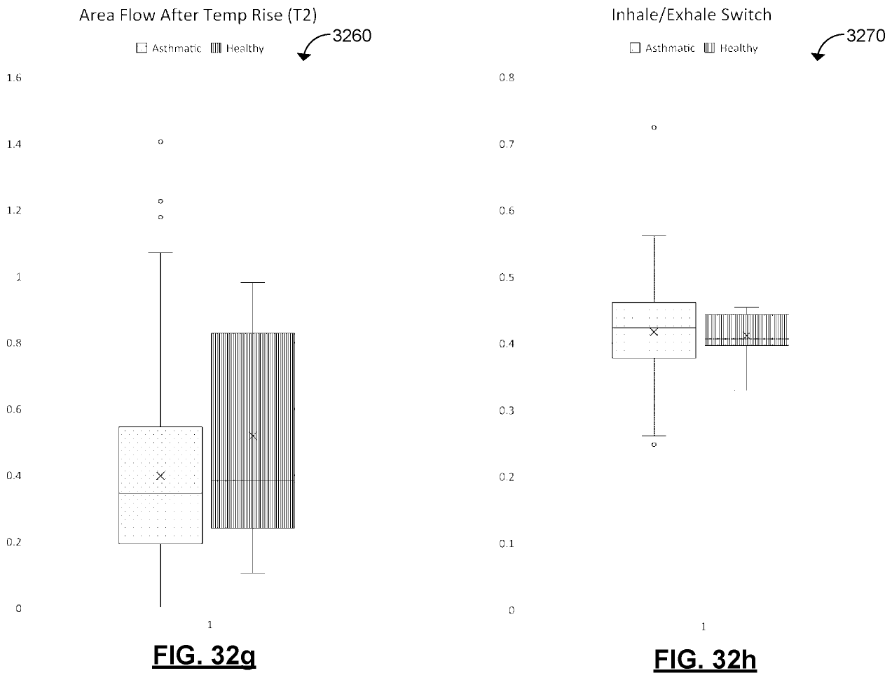

All experiments conducted by healthy individuals were compared to all experiments performed by asthmatic individuals with an average symptom rating greater than 1. Mean and standard deviation of each feature for all experiments was calculated, and statistical significance was determined for the mean of the two groups. Results can be seen in Tables 7a, 7b and 7c (above) and FIGS. 32a, 32b, 32c, 32d, 32e, 32f, 32g and 32h.

Significant features include: Selected Breath Max Temp, Time to CO2 Increase, Breathing Rate, Area Flow after CO2 Rise, Area Flow after Temp Rise, V1/V2, V1/Vtotal, V2/Vtotal

### Conclusions

Asthma symptoms and control is able to be detected using the multivariate spirometer, using the breath flow, CO2, and temperature variables.

There is some distinction between healthy individuals and asthmatic individuals, as well as healthy individuals and symptomatic asthmatic individuals.

### Methods

### Statistical Analysis

Student's t-test was performed (2 tailed, unequal variance) with a significance value of p<0.05.

### Automatic Breath Selection

Breaths were selected from the flow data. A flow of zero means that the individual is not inhaling or exhaling. All locations of positive flow, negative flow, and zero flow are determined. A breath is defined as subsequent zero flow, negative flow, zero flow, positive flow, zero flow (corresponding to one inhale and one exhale).

For the purposes of this analysis, the second breath from each individual will be the automatically selected breath.

### V1 and V2

Biologically active air is the air that makes it to the alveoli in the lungs for gas exchange. This volume of air will be defined as V2. However, not all inhaled air makes it to the alveoli (some is just present in the bronchi/bronchioles/trachea—without which these structures may collapse). The air that does not make it to the alveoli will be defined as V1. Total air inhaled or exhaled (Vtotal) is the sum of V1 and V2.

V1 is determined by integrating the flow between the start of exhalation and the time CO2 levels begin to increase. Vtotal is determined by integrating the entire exhalation. V2 is determined by subtracting V1 from Vtotal.

### Temperature Increase and CO2 Increase

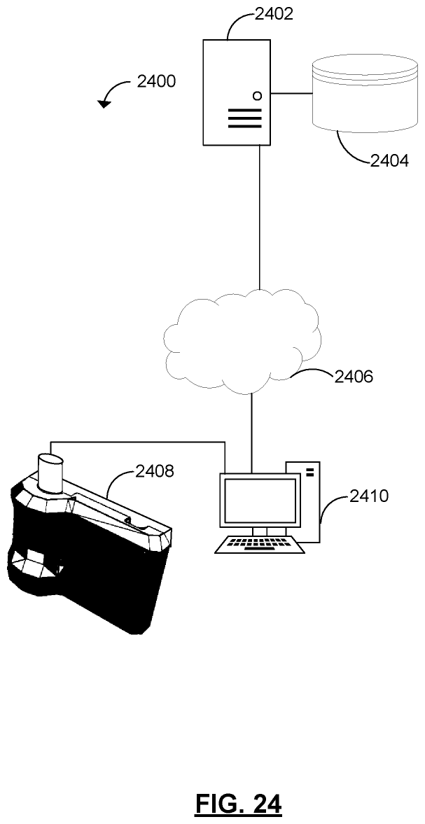

There is a delay between when the flow becomes positive (i.e. when the exhalation begins) and when the temperature and CO2 begin to decrease. This effect can be seen below, using the CO2 rise as an example. Referring to FIG. 28, flow is shown at the beginning of exhalation 2802, and CO2 can be seen including the beginning of CO2 rise 2804. The delay between when the flow becomes positive (i.e. when the exhalation begins) and when the temperature and CO2 begin to decrease was measure based on the beginning of exhalation 2802 and the beginning of CO2 rise 2804.

### Features

### Entire Time Series

Proportion of Time Exhaling: Proportion of the spirometer data that the individual spent exhaling. Calculated by dividing the time there is positive flow by the total time of the recording.

CO2 Rate of Change: Calculated by first taking the derivative of the entire time series. The average of the largest 10% of values of positive rates of change was taken (denoted: highest). This was repeated using the largest 10% of the absolute values of the negative rates of change (denoted: lowest). Final value is calculated by taking the absolute value of the quotient of the average highest positive and negative values (i.e. absolute value of highest/lowest). This value is less than 1 in uncontrolled individuals, indicating that the inhale rate of change is less than the exhale rate of change of CO2 for uncontrolled asthmatics.

Breathing Rate: Inverse of the average breath length; 1/(Average breath length in minutes).

### Single Extracted Breath

Single Breath Max Temp: Maximum temperature for a single extracted breath.

Time to Temperature Increase: Time from the start of the breath until the temperature begins to increase.

Time to CO2 Increase: Time from the start of the breath until the CO2 begins to increase.

Time to CO2 Increase—Time to Temp Increase: Difference between the time of the CO2 increase and the time of the temperature increase.

Area Flow Before CO2 Increase (V1): Represents the volume of air exhaled before the CO2 starts to increase. Calculated by taking the integral of the flow from the start of exhalation to the time the CO2 begins to increase.

Area Flow After CO2 Increase (V2): Represents the volume of air exhaled after the CO2 starts to increase. Calculated by taking the integral of the flow from the time the CO2 begins to increase to the end of the exhalation.

Area Flow Before Temperature Increase (T1): Represents the volume of air exhaled before the temperature starts to increase. Calculated by taking the integral of the flow from the start of exhalation to the time the temperature begins to increase.

Area Flow After Temperature Increase (T2): Represents the volume of air exhaled after the temperature starts to increase. Calculated by taking the integral of the flow from the time the temperature begins to increase to the end of the exhalation.

Area After CO2 Rise*Area After Temp Rise: Multiply V2*T2

Inhale/Exhale Switch: Proportion of time through the selected breath that the individual switches to exhaling. If the individual is exhaling 60% of the breath, switch will occur 40% through the breath.

V1/V2: Ratio of the volume of air exhaled prior to CO2 increase and the volume of air exhaled after the CO2 increase.

V1/Vtotal: Proportion of the total exhaled volume that was exhaled before the CO2 began to increase.

V2/Vtotal: Proportion of the total exhaled volume that was exhaled after the CO2 began to increase.

T1/T2: Ratio of the volume of air exhaled prior to temperature begins to increase and the volume of air exhaled after the temperature begins to increase.

T1/Ttotal: Proportion of the total exhaled volume that was exhaled before the temperature began to increase.

T2/Ttotal: Proportion of the total exhaled volume that was exhaled after the temperature began to increase.

We claim:

1. A spirometer comprising:
a housing comprising a first opening in fluid communication with a second opening, the first opening and second opening defining a flow path;
one or more sensors selected from the group of:
a carbon dioxide (CO₂) sensor for detecting CO₂ concentration values of air in the flow path;
a temperature sensor for detecting temperature of air in the flow path; and
a flow sensor for determining flow rates of air in the flow path.

2. The spirometer of claim 1, comprising:
two or more sensors selected from the group of:
the carbon dioxide (CO₂) sensor for detecting CO₂ concentration values of air in the flow path;
the temperature sensor for detecting temperature of air in the flow path; and
the flow sensor for determining flow rates of air in the flow path.

3. The spirometer of claim 2, comprising:
the carbon dioxide (CO₂) sensor for detecting CO₂ concentration values of air in the flow path;
the temperature sensor for detecting temperature of air in the flow path; and
the flow sensor for determining flow rates of air in the flow path.

4. The spirometer of claim 3, wherein the flow path is linear.

5. The spirometer of claim 3, where the flow path is non-linear.

6. The spirometer of claim 4, wherein the one or more sensors detect a plurality of CO₂ concentration values, a plurality of temperatures and/or a plurality of flow rates during inhalation of air by a subject through the flow path.

7. The spirometer of claim 4, wherein the one or more sensors detect a plurality of CO₂ concentration values, a plurality of temperatures and/or a plurality of flow rates during exhalation of air by a subject through the flow path.

8. The spirometer of claim 7, wherein the one or more sensors detects the plurality of CO₂ concentration values, the plurality of temperatures, and/or the plurality of flow rates as the subject continuously breathes through the flow path.

9. The spirometer of claim 8, wherein the first opening is configured to detachably receive a mouthpiece.

10. The spirometer of claim 9, wherein the mouthpiece is shaped to form a substantially airtight seal with the lips of a subject using the spirometer.

11. The spirometer of claim 10, wherein the mouthpiece has a circular or oval cross section.

12. The spirometer of claim 11, wherein the mouthpiece is made of a plastic material that is biodegradable at a temperature above 50 degrees Celsius.

13. The spirometer of claim 12 further comprising a laminarization means disposed between the first opening and the second opening in order to produce a laminar airflow through the flow path.

14. The spirometer of claim 12, wherein the temperature sensor comprises a temperature sensing member in the flow path proximate to the first opening relative to the CO₂ sensor or the flow sensor, optionally relative to a CO₂ sensing member or a flow sensing member.

15. The spirometer of claim 14, wherein the one or more sensors detect CO₂ concentration values, temperatures and/or flow rates at a predetermined frequency of at least 3, 4, 5, 6, 7, 8, 9, 10, 20, 25, 30, 40 or 50 times per second.

16. The spirometer of claim 15, wherein the temperature sensor has a resolution of at least 0.5, 0.25, 0.1, 0.08, 0.06, 0.05 or 0.04 degrees Celsius.

17. The spirometer of claim 16, wherein the flow sensor is a bidirectional flow sensor.

18. The spirometer of claim 1, wherein:
the carbon dioxide (CO₂) sensor comprises a metal oxide gas sensor for detecting volatile organic compounds (VOCs), optionally an ams® Digital Gas Sensor;
the temperature sensor comprises a band gap temperature sensor; and/or
the flow sensor comprises thermal mass flow sensor element.

19. The spirometer of claim 1, further comprising:
a real-time clock; and
wherein the one or more sensors generate a plurality of sensor readings timestamped with an output of the real-time clock.

20. The spirometer of claim 1, further comprising:
a processor for generating one or more output readings based on the one or more CO₂ concentration values, temperatures and/or flow rates detected by the one or more sensors.

21. The spirometer of claim 20, further comprising:
memory for storing the one or more CO₂ concentration values, temperatures and/or flow rates detected by the one or more sensors, optionally for storing the one or more output readings.

22. The spirometer of claim 21, further comprising a display for presenting the output readings to a user.
